SEQUENCE LISTING (5 sequences)

SEQ ID NO: 1 - DNA, 867 bp, Homo sapiens
  CDS (272)..(799)
  sig_peptide (272)..(325)
gccgccagtgtgatggatatctgcagaattcgccctttgaaacaggctcctaggagaccagaagcagcagcctttcctgagctcagtgcctgctgttccaggccttacctgctgggcactaacggcggagccaggatggggacagaataaaggagccacgacctgtgccaccaactcgcactcagactctgaactcagacctgaaatcttctcttcacgggaggcttggcagtttttcttactcctgtggtctccagatttcaagcctaagatgaaagcctctagtcttgccttcagccttctctctgctgcgttttatctcctatggactccttccactggactgaagacactcaatttgggaagctgtgtgatcgccacaaaccttcaggaaatacgaaatggattttctgagatacggggcagtgtgcaagccaaagatggaaacattgacatcagaatcttaaggaggactgagtctttgcaagacacaaagcctgcgaatcgatgctgcctcctgcgccatttgctaagactctatctggacagggtatttaaaaactaccagacccctgaccattatactctccggaagatcagcagcctcgccaattcctttcttaccatcaagaaggacctccggctctgtcatgcccacatgacatgccattgtggggaggaagcaatgaagaaatacagccagattctgagtcactttgaaaagctggaacctcaggcagcagttgtgaaggctttgggggaactagacattcttctgcaatggatggaggagacagaataggaggaaagtgatgctgctgctaagaatattcgaggtcaaagggcgaattccagcacactggcggc

SEQ ID NO: 2 - protein, 176 aa, Homo sapiens
MKASSLAFSLLSAAFYLLWTPSTGLKTLNLGSCVIATNLQEIRNGFSEIRGSVQAKDGNIDIRILRRTESLQDTKPANRCCLLRHLLRLYLDRVFKNYQTPDHYTLRKISSLANSFLTIKKDLRLCHAHMTCHCGEEAMKKYSQILSHFEKLEPQAAVVKALGELDILLQWMEETE

SEQ ID NO: 3 - protein, 176 aa, Homo sapiens
  MISC_FEATURE (48)..(48): Xaa= Asp or Glu
MKASSLAFSLLSAAFYLLWTPSTGLKTLNLGSCVIATNLQEIRNGFSXIRGSVQAKDGNIDIRILRRTESLQDTKPANRCCLLRHLLRLYLDRVFKNYQTPDHYTLRKISSLANSFLTIKKDLRLXHAHMTCHCGEEAMKKYSQILSHFEKLEPQAAVVKALGELDILLQWMEETE

SEQ ID NO: 4 - DNA, 38 bp, artificial
  synthetic construct
ttggcgcgccatccaccatgaaagcctctagtcttgcc

SEQ ID NO: 5 - DNA, 60 bp, Artificial
  synthetic construct
tagcggccgctacttgtcgtcgtcatccttgtagtcttctgtctcctccatccattgcag

(19) United States
(12) Patent Application Publication
(10) Pub. No.: US 2004/0162237 A1
(43) Pub. Date: Aug. 19, 2004

(54) Use of lp82 to treat body weight disorders

(76) Inventor: Niles Wayne Fox, Indianapolis, IN (US)

Correspondence Address: MaryAnn Wiskerchen, Eli Lilly & Company, Patent Division, PO Box 6288, Indianapolis, IN, 46206-6288, US

(21) Appl. No.: 10/466,695

(22) PCT Filed: Jan. 15, 2002
(86) PCT No.: PCT/US02/00498

(51) Int. Cl. A61K 38/17; A61K 39/395

(52) U.S. Cl. 514/12; 424/145.1

(57) ABSTRACT

The present invention relates to a mammalian gene that is involved in the control of mammalian body weight. More specifically the present invention relates to methods of using compositions comprising LP82 polynucleotides, LP82 polypeptides, and/or LP82 antibodies for the prevention and/or treatment of mammalian body weight disorders, including obesity, cachexia, and anorexia.

### CROSS-REFERENCE

This application claims the benefit of U.S. Provisional Application No. 60/264,239, filed Jan. 26, 2001.

### FIELD OF THE INVENTION

The present invention relates to recombinant DNA technology as applied to the field of human medicine. In particular, the invention relates to new methods of treating or preventing mammalian body weight disorders including, but not limited to, obesity that comprise the administration of LP82, a recently identified interleukin like polypeptide. Additionally, new methods of treating or preventing mammalian body weight disorders including, but not limited to, cachexia, bulimia, and anorexia that comprise the administration of antibodies and other agents that neutralize or antagonize LP82 activity are disclosed herein.

### BACKGROUND OF THE INVENTION

Obesity continues to remain a serious health concern in modern society. Generally defined as an excess of body fat relative to lean body mass, obesity has been estimated to affect 34% of all Americans; that is, their body weight exceeds desirable body weight by 20% or more. In addition to being a cause of psychological distress and depression, obesity is responsible for increased incidence of diseases such as coronary artery disease, hypertension, stroke, diabetes, hyperlipidemia, and some cancers (See, e.g., Nishina, P. M. et al., 1994, Metab. 43: 554-558; Grundy, S. M. & Barnett, J. P., 1990, Dis. Mon. 36: 641-731).

Numerous studies have demonstrated that reduction in obesity by diet and exercise reduces these complications dramatically. Unfortunately, these treatments are largely unsuccessful, failing at a rate approximating 95%.

Obesity is not merely a behavioral problem, i.e., the result of voluntary hyperphagia. Rather, the differential body composition observed between obese and normal subjects results from differences in both metabolism and neurological interactions. These differences seem to be, to some extent, due to differences in gene expression, and/or level of gene products or activity (Friedman, J. M. et al., 1991, Mammalian Gene 1: 130-144) that can contribute to increased appetite, preference for highly caloric foods, reduced physical activity, and increased lipogenic metabolism. As a result many people inheriting certain genetic traits are prone to becoming obese in spite of their efforts to combat the condition. Oftentimes in obese subjects food intake and energy expenditure do not come into balance until there is excess adipose tissue. While a combination of a reasonable diet and exercise is best, attempts to reduce food intake, or to decrease hyper-nutrition, are usually fruitless in the medium term because the weight loss induced by dieting results in both increased appetite and decreased energy expenditure. (Leibel et al., New England Journal of Medicine 322:621-28, (1995)). The craving for food while on a diet can be quite acute and the urge to stray from the dietary regimen constant. Furthermore, the intensity of physical exercise required to expend enough energy to materially lose adipose mass is too great for many obese people to undertake on a sufficiently frequent basis.

Thus, obesity is currently a poorly treatable, chronic, and essentially intractable metabolic disorder. Considering the high prevalence of obesity in our society and the serious consequences associated therewith, there is a need in the art for therapeutic agents that reduce total body weight of obese subjects without significant adverse side effects and that may help the obese subject maintain the reduced weight level. Accordingly, it is an object of the present invention to provide methods of treatment that are useful in returning the body weight of obese subjects toward a normal, ideal body weight. Other objects of the present invention include providing a therapy for obesity that can correct a predisposition toward obesity, maintain a lower body weight for an extended period of time and specifically address the carbohydrate craving universally complained of by individuals on a diet, as well as generally increase the well being and vigor among these affected individuals.

### SUMMARY OF THE INVENTION

A new member of the interleukin family was described in international patent applications WO99/27103 and WO 00/12708 (the contents of each are incorporated herein by reference) and alternatively named human Zcyto10 and PRO1801, respectively. This protein, hereinafter referred to as LP82, exhibits the four alpha helix structure typical of the known cytokines and shares significant homology with IL-10 and IL-19. Applicant's have discovered that over-expression of LP82 in transgenic mice leads to significant alterations in body weight and body composition in response to various diets. The studies disclosed herein indicate that over-expression of LP82 in mice is accompanied by altered energy expenditure and body weight composition in these rodents. The thermogenic activity attributable to LP82 activity may be useful in reducing, or preventing the development of excess adipose tissue, such as that found in obesity. (Harrison's Principles of Internal Medicine 12th Edition, McGraw Hill, Inc. (199 1) p. 411). An increased level of thermogenesis induced by administration of LP82 polypeptides to obese patients, or patients with a predisposition toward obesity, may increase energy expenditure, reduce the level of adipose tissue, and thereby help the patient maintain a desired body weight and avoid the complications associated with obesity.

The present invention therefore provides methods of treating or preventing body weight disorders including, but not limited to, obesity and disorders commonly associated with obesity comprising the administration of agonists of LP82 activity. Methods of treating or preventing body weight disorders including, but not limited to, cachexia, bulimia, anorexia, and disorders commonly associated with such disorders comprising the administration of antagonists of LP82 activity is also contemplated by the present invention. LP82 agonists and antagonists may include, for example, small molecules, LP82 polypeptides, as defined herein, and antibodies directed against the LP82 polypeptide. Agonists and antagonists of the invention also include nucleotide sequences, such as antisense and ribozyme molecules, and gene or regulatory sequence replacement constructs, that can be used to inhibit or enhance expression of the LP82 gene.

### BRIEF DESCRIPTION OF THE FIGURES

FIG. 1 displays the average fuel intake and utilization by wild type and LP82 transgenic mice 27 days after a diet comprised of 19% protein, 40% fat and 41% carbohydrate.

FIG. 2 displays the average fuel intake and utilization by wild type and LP82 transgenic mice 43 days after a diet comprised of 19% protein, 40% fat and 41% carbohydrate.

FIG. 3 displays the average amount of lean mass and fat mass in wild type and LP82 transgenic mice 43 days after a diet comprised of 19% protein, 40% fat and 41% carbohydrate.

FIG. 4 displays the average fuel intake and utilization by wild type and LP82 transgenic mice 7 days after a diet comprised of 22% protein, 5% fat, and 73% carbohydrate.

FIG. 5 displays the average fuel intake and utilization by wild type and LP82 transgenic mice 14 days after a diet comprised of 22% protein, 5% fat, and 73% carbohydrate.

FIG. 6 displays the average amount of lean mass and fat mass in wild type and LP82 transgenic mice 14 days after a diet comprised of 22% protein, 5% fat, and 73% carbohydrate.

FIG. 7 displays the average fuel intake and utilization by wild type and LP82 transgenic mice 20 days after a diet comprised of 40% protein, 40% fat and 20% carbohydrate.

FIG. 8 displays the average fuel intake and utilization by wild type and LP82 transgenic mice 35 days after a diet comprised of 40% protein, 40% fat and 20% carbohydrate.

FIG. 9 displays the average amount of lean mass and fat mass in wild type and LP82 transgenic mice 35 days after a diet comprised of 40% protein, 40% fat and 20% carbohydrate.

FIG. 10 represents map of plasmid used to generate LP82-transgenic mice.

### DETAILED DESCRIPTION OF THE INVENTION

Definitions

The term “amino acid” is used herein in its broadest sense, and includes naturally occurring amino acids as well as non-naturally occurring amino acids, including amino acid variants and derivatives. The latter includes molecules containing an amino acid moiety. One skilled in the art will recognize, in view of this broad definition, that reference herein to an amino acid includes, for example, naturally occurring proteogenic L-amino acids; D-amino acids; chemically modified amino acids such as amino acid variants and derivatives; naturally occurring non-proteogenic amino acids such as norleucine, β-alanine, ornithine, etc.; and chemically synthesized compounds having properties known in the art to be characteristic of amino acids.

The incorporation of non-natural amino acids, including synthetic non-native amino acids, substituted amino acids, or one or more D-amino acids into the LP82 polypeptides and/or fragments thereof and/or variants thereof of the present invention (“D-LP82 polypeptides”) is advantageous in a number of different ways. D-amino acid-containing polypeptides, etc., exhibit increased stability in vitro or in vivo compared to L-amino acid-containing counterparts. Thus, the construction of LP82 polypeptides, etc., incorporating D-amino acids can be particularly useful when greater stability is desired or required in vivo. More specifically, D-peptides, etc., are resistant to endogenous peptidases and proteases, thereby providing improved bioavailability of the molecule, and prolonged lifetimes in vivo when such properties are desirable. When it is desirable to allow the peptide, etc., to remain active for only a short period of time, the use of L-amino acids therein will permit endogenous peptidases, proteases, etc., to digest the molecule, thereby limiting the cell's exposure to the molecule. Additionally, D-peptides, etc., cannot be processed efficienty for major histocompatibility complex class II-restricted presentation to T helper cells, and are therefore less likely to induce humoral immune responses in the whole organism.

In addition to using D-amino acids, those of ordinary skill in the art are aware that modifications in the amino acid sequence of a LP82 polypeptide and/or any fragment thereof and/or any variant thereof can result in functional LP82 polypeptides that display equivalent or superior functional characteristics when compared to the original polypeptide sequence as shown in SEQ ID NO:2, or any fragment thereof. Thus, the methods of the present invention contemplate alterations in the LP82 polypeptides and/or fragments thereof and/or variants thereof that may include one or more amino acid insertions, deletions, substitutions, truncations, fusions, shuffling of subunit sequences, and the like, either from natural mutations or human manipulation, provided that the sequences produced by such modifications have substantially the same (or improved or reduced, as may be desirable) activity(ies) as the LP82 polypeptide and/or fragment thereof and/or variant thereof disclosed herein.

The term “LP82 antagonist” is used in the broadest sense and includes any molecule that partially or fully blocks, inhibits, or neutralizes a biological activity of a LP82 polypeptide as defined herein. In a similar manner, the term “LP82 agonist” is used in the broadest sense and includes any molecule that induces or increases the expression and/or biological activity of any naturally occurring LP82 polypeptide. Suitable LP82 agonists or LP82 antagonists include agonistic or antagonistic antibodies or antibody fragments, LP82 fragments, LP82 variants as defined herein, LP82 directed ribozymes, LP82 antisense nucleic acids, and small organic molecules. Methods for identifying such LP82 agonists or LP82 antagonists may comprise contacting an LP82 polypeptide with a candidate LP82 agonist or LP82 antagonist molecule and measuring a detectable change in one or more biological activities normally associated with the LP82 polypeptide.

“Antibodies” (Abs) and “immunoglobulins” (Igs) are glycoproteins having the same structural characteristics. While antibodies exhibit binding specificity to a specific antigen, immunoglobulins include both antibodies and other antibody-like molecules that lack antigen specificity. Polypeptides of the latter kind are, for example, produced at low levels by the lymph system and at increased levels by myelomas. The term “antibody” is used in the broadest sense and specifically covers, without limitation, intact monoclonal antibodies, polyclonal antibodies, multispecific antibodies (e.g., bispecific antibodies) formed from at least two intact antibodies, and antibody fragments so long as they exhibit the desired biological activity.

“Antibody fragments” comprise a portion of an intact antibody, preferably the antigen binding or variable region of the intact antibody. Examples of antibody fragments include Fab, Fab′, F(ab′)₂ and Fv fragments; diabodies; linear antibodies [Zapata, et al., Protein Engin. 8(10): 1057-62 (1995)]; single-chain antibody molecules; and multispecific antibodies formed from antibody fragments.

“Fv” is the minimum antibody fragment which contains a complete antigen-recognition and binding site. This region consists of a dimer of one heavy- and one light-chain variable domain in tight, non-covalent association. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDR specific for an antigen) has the ability to recognize and bind antigen, although at a lower affinity than the entire binding site.

“Single-chain Fv” or “sFv” antibody fragments comprise the VH and VL domains of antibody, wherein these domains are present in a single polypeptide chain. Preferably, the Fv polypeptide further comprises a polypeptide linker between the VH and VL domain, which enables the sFv to form the desired structure for antigen binding. For a review of sFv, see Pluckthun, The Pharmacology of Monoclonal Antibodies, vol. 113, Rosenburg and Moore, eds., Springer-Verlag, New York, pp. 269-315 (1994).

The term “diabodies” refers to small antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies are described more fully in, for example, EP 404 097, WO 93/11161; and Hollinger, et al., Proc. Natl. Acad. Sci. USA 90: 6444-48 (1993).

An “isolated” antibody is one that has been identified and separated and/or recovered from a component of its natural environment. Contaminant components of its natural environment are materials which would interfere with diagnostic or therapeutic uses for the antibody, and may include enzymes, hormones, and other proteinaceous or non-proteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue, or preferably, silver stain. Isolated antibody includes the antibody in situ within recombinant cells since at least one component of the antibody's natural environment will not be present. Ordinarily, however, isolated antibody will be prepared by at least one purification step.

The terms “complementary” or “complementarity” as used herein refer to the capacity of purine and pyrimidine nucleotides to associate through hydrogen bonding to form double stranded nucleic acid molecules. The following base pairs are related by complementarity: guanine and cytosine; adenine and thymine; and adenine and uracil. As used herein, “complementary” means that the aforementioned relationship applies to substantially all base pairs comprising two single-stranded nucleic acid molecules over the entire length of said molecules. “Partially complementary” refers to the aforementioned relationship in which one of two single-stranded nucleic acid molecules is shorter in length than the other such that a portion of one of the molecules remains single-stranded.

The term “fragment” or “fragment thereof” in reference to a LP82 gene or cDNA sequence, refers to a fragment, or sub-region of an LP82 nucleic acid such that said fragment comprises 15 or more nucleotides that are contiguous in the native nucleic acid molecule as shown in SEQ ID NO:1.

The term “fragment” or “fragment thereof” in reference to a LP82 protein or polypeptide sequence, refers to a fragment, or sub-region of an LP82 protein or polypeptide, such that said fragment comprises 5 or more amino acids that are contiguous in the native polypeptide as shown in SEQ ID NO:2.

The term “functional” in reference to a LP82 molecule is intended to mean that a particular molecule exhibits biological activities, in vivo or in vitro, that are similar or identical to the biological activities attributable to LP82 polypeptides as disclosed herein, i.e., the ability to prevent fat accretion in response to diets rich in fat and/or protein. “Functional fragment” or “functionally-related” as used herein, refers to an isolated sub-region, or fragment of a LP82 polypeptide that, for example, comprises a functionally distinct region such as an active site on an enzyme, or a binding site for a ligand, receptor, polypeptide, or other substrate. Functional fragments may be produced by recombinant DNA methodologies, enzymatic/proteolytic digestions, or as natural products of alternative splicing processes.

“Host cell” refers to any eukaryotic or prokaryotic cell that is suitable for propagating and/or expressing a cloned gene contained on a vector that is introduced into said host cell by, for example, transformation or transfection, or the like.

The term “LP82” refers to a nucleic acid, gene, cDNA (e.g. SEQ ID NO:1) and fragments thereof as well as to any polypeptide sequence (e.g., SEQ ID NO:2) encoded thereby.

The term “LP82” without further limitation refers to both the native LP82 polypeptide (SEQ ID NO:2) as well as the mature form of the LP82 polypeptide which is predicted to be amino acids 20 through 176 of SEQ ID NO:2. If not state otherwise, the term “LP82 polypeptide” encompasses full-length, truncated, secreted, mature, variant, alternatively spliced, and allelic forms of the LP82 polypeptide shown in SEQ ID NO:2.

An “LP82 polypeptide antibody” or “LP82 antibody” refers to an antibody as defined herein that recognizes and binds at least one epitope of a LP82 polypeptide as defined herein.

The term “LP82 variant” as used herein refers to a polynucleotide or polypeptide as shown in SEQ ID NO:1 or 2, respectively, as well any fragment thereof, that further comprises at least one of the various types of modifications contemplated herein. Furthermore, LP82 variant, as applied to a polypeptide, is intended to refer to a “functional” LP82 polypeptide, as defined herein, having at least about 90% amino acid sequence identity with an LP82 polypeptide having the deduced amino acid sequences as shown in SEQ ID NO:2. Such LP82 polypeptide variants include, for instance, LP82 polypeptides or fragments thereof wherein one or more amino acid residues are added, substituted or deleted, at the N- or C-terminus or within the sequence of SEQ ID NO:2. Ordinarily, a LP82 variant will have at least about 90% amino acid sequence identity, preferably at least about 91% sequence identity, yet more preferably at least about 92% sequence identity, yet more preferably at least about 93% sequence identity, yet more preferably at least about 94% sequence identity, yet more preferably at least about 95% sequence identity, yet more preferably at least about 96% sequence identity, yet more preferably at least about 97% sequence identity, yet more preferably at least about 98% sequence identity, yet more preferably at least about 99% amino acid sequence identity with the amino acid sequence as shown in SEQ ID NO:2, or any fragment thereof, with or without the signal peptide.

“LP82 variant” in reference to a polynucleotide is intended to refer to a nucleic acid molecule having at least about 70% nucleic acid sequence identity with the polynucleotide sequence shown in SEQ ID NO:1. Ordinarily, an LP82 polynucleotide variant will have at least about 70% nucleic acid sequence identity, more preferably at least about 80% nucleic acid sequence identity, yet more preferably at least about 81% nucleic acid sequence identity, yet more preferably at least about 82% nucleic acid sequence identity, yet more preferably at least about 83% nucleic acid sequence identity, yet more preferably at least about 84% nucleic acid sequence identity, yet more preferably at least about 85% nucleic acid sequence identity, yet more preferably at least about 86% nucleic acid sequence identity, yet more preferably at least about 87% nucleic acid sequence identity, yet more preferably at least about 88% nucleic acid sequence identity, yet more preferably at least about 89% nucleic acid sequence identity, yet more preferably at least about 90% nucleic acid sequence identity, yet more preferably at least about 91% nucleic acid sequence identity, yet more preferably at least about 92% nucleic acid sequence identity, yet more preferably at least about 93% nucleic acid sequence identity, yet more preferably at least about 94% nucleic acid sequence identity, yet more preferably at least about 95% nucleic acid sequence identity, yet more preferably at least about 96% nucleic acid sequence identity, yet more preferably at least about 97% nucleic acid sequence identity, yet more preferably at least about 98% nucleic acid sequence identity, yet more preferably at least about 99% nucleic acid sequence identity with the nucleic acid sequences shown above.

The term “half-life” as used herein refers to the time required for approximately half of the molecules making up a population of said molecules to be cleaved in vitro. More specifically, “plasma half-life” refers to the time required for approximately half of the molecules making up a population of said molecules to be removed from circulation or be, otherwise, rendered inactive in vivo.

The term “homolog” or “homologous” designates a relationship of partial identity or similarity of sequence between nucleic acid molecules or protein molecules at one or more regions within said molecules.

The term “hybridization” as used herein refers to a process in which a single-stranded nucleic acid molecule joins with a complementary strand through nucleotide base pairing. “Selective hybridization” refers to hybridization under conditions of high stringency. The degree of hybridization depends upon, for example, the degree of homology or relatedness, the stringency of hybridization, and the length of hybridizing strands.

The term “inhibit” or “inhibiting” includes the generally accepted meaning, which includes prohibiting, preventing, restraining, slowing, stopping, or reversing progression or severity of a disease or condition.

“Isolated nucleic acid compound” refers to any specific RNA or DNA molecule, however constructed or synthesized or isolated, which is locationally distinct from its natural location, and which is substantially free of other larger or smaller nucleic acid compounds.

The term “mature protein” or “mature polypeptide” as used herein refers to the form(s) of a protein produced by expression in a mammalian cell. It is generally hypothesized that once export of a growing protein chain across the rough endoplasmic reticulum has been initiated, proteins secreted by mammalian cells have a signal peptide (SP) sequence which is cleaved from the complete polypeptide to produce a “mature” form of the protein. Oftentimes, cleavage of a secreted protein is not uniform and may result in more than one species of mature protein. The cleavage site of a secreted protein is determined by the primary amino acid sequence of the complete protein and generally cannot be predicted with complete accuracy. Methods for predicting whether a protein has an SP sequence, as well as the cleavage point for that sequence, are available. A cleavage point may exist within the N-terminal domain of LP82 between amino acid 10 and amino acid 35. More specifically the cleavage point is likely to exist after amino acid 15 but before amino acid 25, more likely after amino acid 24 but before amino acid 25. As one of ordinary skill would appreciate, cleavage sites sometimes vary from organism to organism and cannot be predicted with absolute certainty. Optimally, cleavage sites for a secreted protein are determined experimentally by amino-terminal sequencing of the one or more species of mature proteins found within a purified preparation of the protein.

A “nucleic acid probe” or “probe” as used herein is a labeled nucleic acid compound that hybridizes with another nucleic acid compound. “Nucleic acid probe” means a single stranded nucleic acid sequence that will combine with a complementary or partially complementary single stranded target nucleic acid sequence to form a double-stranded molecule. A nucleic acid probe may be an oligonucleotide or a nucleotide polymer. A probe will usually contain a detectable moiety which may be attached to the end(s) of the probe or be internal to the sequence of the probe.

The terms “ortholog”, “orthologue”, or “orthologous” refers to two or more genes or proteins from different organisms that exhibit sequence homology. The terms “paralog”, “paralogue” or “paralogous” refers to two or more genes or proteins within a single organism that exhibit sequence homology.

The term “plasmid” refers to an extrachromosomal genetic element. The plasmids disclosed herein are commercially available, publicly available on an unrestricted basis, or can be constructed from readily available plasmids in accordance with published procedures.

A “primer” is a nucleic acid fragment which functions as an initiating substrate for enzymatic or synthetic elongation of, for example, a nucleic acid molecule.

The term “promoter” refers to a nucleic acid sequence that directs transcription, for example, of DNA to RNA. An inducible promoter is one that is regulatable by environmental signals, such as carbon source, heat, or metal ions, for example. A constitutive promoter generally operates at a constant level and is not regulatable.

“Recombinant DNA cloning vector” as used herein refers to any autonomously replicating agent, including, but not limited to, plasmids and phages, comprising a DNA molecule to which one or more additional DNA segments can or have been incorporated.

The term “recombinant DNA expression vector” or “expression vector” as used herein refers to any recombinant DNA cloning vector, for example a plasmid or phage, in which a promoter and other regulatory elements are present thereby enabling transcription of an inserted DNA, which may encode a protein.

A “small molecule” is defined herein to have a molecular weight below about 500 daltons.

The term “stringency” refers to hybridization conditions. High stringency conditions disfavor non-homologous base pairing. Low stringency conditions have the opposite effect. Stringency may be altered, for example, by temperature and salt concentration.

“Low stringency” conditions comprise, for example, a temperature of about 37° C. or less, a formamide concentration of less than about 50%, and a moderate to low salt (SSC) concentration; or, alternatively, a temperature of about 50° C. or less, and a moderate to high salt (SSPE) concentration, for example 1M NaCl.

“High stringency” conditions comprise, for example, a temperature of about 42° C. or less, a formamide concentration of less than about 20%, and a low salt (SSC) concentration; or, alternatively, a temperature of about 65° C., or less, and a low salt (SSPE) concentration. For example, high stringency conditions comprise hybridization in 0.5 M NaHPO₄, 7% sodium dodecyl sulfate (SDS), 1 mM EDTA at 65° C. (Ausubel, F. M. et al. Current Protocols in Molecular Biology, Vol. I, 1989; Green Inc. New York, at 2.10.3).

“SSC” comprises a hybridization and wash solution. A stock 20×SSC solution contains 3 M sodium chloride, 0.3× sodium citrate, pH 7.0.

“SSPE” comprises a hybridization and wash solution. A 1×SSPE solution contains 180 mM NaCl, 9 mM Na₂HPO₄, 0.9 mM NaH₂PO₄ and 1 mM EDTA, pH 7.4.

“Substantially pure,” used in reference to a peptide or protein, means separation from other cellular and non-cellular molecules, including other protein molecules. A substantially pure preparation would be about at least 85% pure; preferably about at least 95% pure. A “substantially pure” protein can be prepared by a variety of techniques, well known to the skilled artisan, including, for example, the IMAC protein purification method.

The term “treatment” or “treating” as used herein describes the management and care of a patient for the purpose of combating a disease, condition, or disorder and includes the administration of a LP82 agonist, LP82 antagonist, LP82 polynucleotide, LP82 polypeptide, LP82 variant, and/or LP82 antibody to prevent the onset of the symptoms or complications, alleviating the symptoms or complications, or eliminating the disease, condition, or disorder. Treating as used herein also includes the administration of the protein for cosmetic purposes.

The term “vector” as used herein refers to a nucleic acid compound used for introducing exogenous or endogenous DNA into host cells. A vector comprises a nucleotide sequence which may encode one or more protein molecules. Plasmids, cosmids, viruses, and bacteriophages, in the natural state or which have undergone recombinant engineering, are examples of commonly used vectors.

The various restriction enzymes disclosed and described herein are commercially available and the manner of use of said enzymes including reaction conditions, cofactors, and other requirements for activity are well known to one of ordinary skill in the art. Reaction conditions for particular enzymes were carried out according to the manufacturer's recommendation.

Example 3 presented below presents data demonstrating that LP82 activity is closely associated with diet-induced metabolic processes, therefore supporting the use of a LP82 agonist, LP82 antagonist, LP82 polynucleotide, LP82 polypeptide, LP82 fragment, LP82 variant, and/or LP82 antibody as a therapeutic for treatment of diet-induced body weight disorders. Applicants have discovered that male and female mice over-expressing LP82 polypeptides react to dietary changes in a manner that prevents accretion of body fat. It was additionally observed that LP82 appears to coordinate with reproductive hormones since a sexual dimorphism was observed in transgenic mice over-expressing LP82. Female mice over-expressing LP82 resisted fat gain when they were offered a diet rich in fat and carbohydrate. Male transgenic mice over-expressing LP82 not only resisted fat accretion but also lost fat when placed on a diet rich in fat and protein. Thus, treatment with a LP82 agonist, LP82 polypeptide, and/or LP82 variant may be useful in preventing gains in body fat in individuals who are subjected to dietary changes. The changes in diet may be the result of medical necessity. For instance, LP82 polypeptides, LP82 variants, and/or LP82 agonists would be useful in preventing fat accretion in individuals that are placed on high energy diets in order to treat cachexia, AIDS, wasting, or frailty. Administration of LP82 agonists, LP82 polypeptides, and/or LP82 variants may also be useful to prevent weight gain after smoking cessation or in those prone to binge eating, particularly in women that crave fat and carbohydrate and men who crave protein and fat, for example. Furthermore, popular diets that promote fat and protein consumption may be more effective at increasing lean mass in males if they were followed in combination with administration of at least one LP82 agonist, LP82 polypeptide, and/or LP82 variant.

The present invention therefore provides methods of treating or preventing obesity, cachexia, bulimia, anorexia, and disorders associated with such conditions that comprise the administration, to a mammal in need of such treatment, of a therapeutically effective amount of a pharmaceutical composition comprising at least one LP82 agonist, LP82 antagonist, LP82 polynucleotide, LP82 polypeptide, LP82 fragment, LP82 variant, and/or LP82 antibody.

A preferred embodiment of the present invention provides methods of treating or preventing body weight disorders including, but not limited to, obesity and disorders commonly associated with obesity comprising the administration to a mammal in need of such treatment a therapeutically effective amount of a pharmaceutical composition comprising an isolated LP82 polypeptide comprising at least one fragment or domain of the polypeptide as shown in SEQ ID NO:2.

The present invention also provides methods of treating or preventing obesity and/or disorders associated with obesity comprising the administration of a therapeutically effective amount of a pharmaceutical composition comprising an LP82 variant as defined herein to a mammal in need of such treatment.

The present invention also provides methods of treating or preventing obesity and/or disorders associated with obesity comprising the administration of a therapeutically effective amount of a pharmaceutical compositions comprising an isolated LP82 polypeptide, LP82 fragment, and/or LP82 variant additionally fused to a heterologous polypeptide to a mammal in need of such treatment.

The present invention also provides methods of treating or preventing body weight disorders including, but not limited to, cachexia, bulimia, and anorexia as well as disorders commonly associated with these conditions comprising the administration of a therapeutically effective amount of a pharmaceutical composition comprising an LP82 antagonist, LP82 agonist, LP82 polynucleotide, LP82 polypeptide, LP82 antibody, and/or LP82 variant to a mammal in need of such treatment.

A preferred embodiment of the present invention provides a method of treating or preventing body weight disorders including, but not limited to, cachexia, bulimia, anorexia, and/or disorders commonly associated with these conditions, comprising the administration of a therapeutically effective amount of a pharmaceutical composition comprising a LP82 antibody to a mammal in need of such treatment.

The present invention further provides a pharmaceutical formulation that comprises at least one LP82 agonist, LP82 antagonist, LP82 polynucleotide, LP82 polypeptide, LP82 fragment, LP82 variant, and/or LP82 antibody together with one or more pharmaceutically acceptable diluents, carriers, or excipients therefor.

The present invention also provides methods of treating or preventing obesity, cachexia, bulimia, anorexia, and disorders associated with such conditions which comprise administering to a mammal in need thereof an therapeutically effective amount of a pharmaceutical composition comprising an isolated polypeptide comprising at least one fragment or domain that is at least 80% identical to the corresponding region of contiguous amino acids as shown in SEQ ID NO:2.

The present invention also provides a method of treating or preventing body weight disorders including, but not limited to, cachexia, bulimia, anorexia, and/or disorders commonly associated with these conditions comprising the administration to a mammal in need thereof a therapeutically effective amount of a composition comprising a LP82′ agonist, LP82 antagonist, LP82 polynucleotide, LP82 polypeptide, LP82 variant, and/or LP82 antibody wherein said composition has at least one activity, such as, but not limited to, inducing weight loss, suppressing appetite, preventing fat accretion, or inhibiting weight gain. An LP82 polypeptide can thus be screened for a corresponding activity according to these effects.

The present invention also provides a method of treating or preventing body weight disorders including, but not limited to, cachexia, bulimia, anorexia, and/or disorders commonly associated with these conditions, comprising the administration to a mammal in need thereof a therapeutically effective amount of a composition comprising a LP82 agonist, LP82 antagonist, LP82 polynucleotide, LP82 polypeptide, LP82 variant, and/or LP82 antibody and a carrier or diluent wherein said composition has at least one activity, such as, but not limited to, inducing weight loss, suppressing appetite, preventing fat accretion, or inhibiting weight gain. The carrier or diluent can optionally be pharmaceutically acceptable, according to known methods.

In another embodiment of the present invention methods are provided for treating or preventing body weight disorders including, but not limited to, cachexia, bulimia, anorexia, and/or disorders commonly associated with these conditions wherein said disorders commonly associated with these conditions include, but are not limited to, dyslipidemia, hypertension, diabetes, and insulin resistance.

The invention further provides for the use of a LP82 agonist, LP82 antagonist, LP82 polypeptide, LP82 nucleic acid, or LP82 antibody in the manufacture of a medicament for the treatment or prevention of obesity, cachexia, bulimia, anorexia, and disorders associated with such conditions.

The present invention contemplates the use of LP82 agonists, LP82 antagonists, LP82 polynucleotides, LP82 polypeptides, LP82 variants, and LP82 antibodies to treat metabolic diseases such as obesity, cachexia, bulimia, anorexia, and/or disorders commonly associated with these conditions such as dyslipidemia and diabetes, especially non-insulin dependent diabetes. Those skilled in the art are able to recognize the presence of such diseases in a subject (e.g, a mammalian subject, such as a human, a domesticated animal, or an animal used in agriculture). Accordingly, the present invention includes the administration of LP82 agonists, LP82 antagonists, LP82 polynucleotides, LP82 polypeptides, LP82 variants, and/or LP82 antibodies for veterinary or human therapeutic uses.

The novel methods contemplated by the present invention are intended to include methods of using LP82 polynucleotides and LP82 polypeptides as shown in SEQ ID NO:1 and 2, respectively, and fragments thereof, as well as LP82 polynucleotide variants or LP82 polypeptide variants that further comprise one or more substitutions, deletions, insertions, inversions, additions, or changes in glycosylation sites or patterns yet have substantially similar biological activities and/or pharmaceutically desired properties as the corresponding unmodified LP82 polynucleotide or LP82 polypeptide.

In one embodiment of the present invention, a single amino acid change is made within the LP82 polypeptide as shown in SEQ ID NO:2, or any fragment thereof. Alternatively, at least two changes are made within at least one of these polypeptide sequences; alternatively, at least three changes are made within at least one of these polypeptide sequences; alternatively, at least four changes up to at least 50 changes are made within at least one of these polypeptide sequences. As the skilled artisan understands, many substitutions, and/or other changes to a protein's sequence or structure, can be made without substantially affecting the biological activity or characteristics of the polypeptide. For example, making conservative amino acid substitutions, or changing one amino acid for another from the same class of amino acids, for example, negatively charged residues, positively charged residues, polar uncharged residues, and non-polar residues, or any other classification acceptable in the art, are often made without significant effects upon function. Modifications of the LP82 polypeptide as shown in SEQ ID NO:2 or fragments thereof, made in accordance with Table I are expected to result in LP82 variants that retain the same or substantially similar biological activity as the unmodified LP82 polypeptide or corresponding fragment thereof, based on art recognized substitutability of certain amino acids (See e.g. M. Dayhoff, In Atlas of Protein Sequence and Structure, Vol. 5, Supp. 3, pgs 345-352, 1978) and are also contemplated by the present invention.

| ORIGINAL RESIDUE | EXEMPLARY SUBSTITUTIONS |
|---|---|
| ALA | SER, THR |
| ARG | LYS |
| ASN | HIS, SER |
| ASP | GLU, ASN |
| CYS | SER |
| GLN | ASN, HIS |
| GLU | ASP, GLU |
| GLY | ALA, SER |
| HIS | ASN, GLN |
| ILE | LEU, VAL, THR |
| LEU | ILE, VAL |
| LYS | ARG, GLN, GLU, THR |
| MET | LEU, ILE, VAL |
| PHE | LEU, TYR |
| SER | THR, ALA, ASN |
| THR | SER, ALA |
| TRP | ARG, SER |
| TYR | PHE |
| VAL | ILE, LEU, ALA |
| PRO | ALA |

One factor that can be considered in making such changes is the hydropathic index of amino acids. The importance of the hydropathic amino acid index in conferring interactive biological function on a protein has been discussed by Kyte and Doolittle (1982, J. Mol. Biol., 157: 105-132). It is accepted that the relative hydropathic character of amino acids contributes to the secondary structure of the resultant protein. This, in turn, affects the interaction of the protein with molecules such as enzymes, substrates, receptors, ligands, DNA, antibodies, antigens, etc. Based on its hydrophobicity and charge characteristics, each amino acid has been assigned a hydropathic index as follows: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate/glutamine/aspartate/asparagine (−3.5); lysine (−3.9); and arginine (−4.5).

As is known in the art, certain amino acids in a peptide, polypeptide, or protein can be substituted for other amino acids having a similar hydropathic index or score and produce a resultant peptide, etc., having similar biological activity, i.e., which still retains biological functionality. In making such changes, it is preferable that amino acids having hydropathic indices within ±2 are substituted for one another. More preferred substitutions are those wherein the amino acids have hydropathic indices within ±1. Most preferred substitutions are those wherein the amino acids have hydropathic indices within ±0.5.

Like amino acids can also be substituted on the basis of hydrophilicity. U.S. Pat. No. 4,554,101 discloses that the greatest local average hydrophilicity of a protein, as governed by the hydrophilicity of its adjacent amino acids, correlates with a biological property of the protein. The following hydrophilicity values have been assigned to amino acids: arginine/lysine (+3.0); aspartate/glutamate (+3.0±1); serine (+0.3); asparagine/glutamine (+0.2); glycine (0); threonine (−0.4); proline (−0.5+1); alanine/histidine (−0.5); cysteine (−1.0); methionine (−1.3); valine (−1.5); leucine/isoleucine (−1.8); tyrosine (−2.3); phenylalanine (−2.5); and tryptophan (−3.4). Thus, one amino acid in a peptide, polypeptide, or protein can be substituted by another amino acid having a similar hydrophilicity score and still produce a resultant peptide, etc., having similar biological activity, i.e., still retaining correct biological function. In making such changes, amino acids having hydropathic indices within ±2 are preferably substituted for one another, those within ±1 are more preferred, and those within ±0.5 are most preferred.

As outlined above, amino acid substitutions in a LP82 polypeptide can be based on the relative similarity of the amino acid side-chain substituents, for example, their hydrophobicity, hydrophilicity, charge, size, etc. Exemplary substitutions that take various of the foregoing characteristics into consideration in order to produce conservative amino acid changes resulting in silent changes within the present peptides, etc., can be selected from other members of the class to which the naturally occurring amino acid belongs. Amino acids can be divided into the following four groups: (1) acidic amino acids; (2) basic amino acids; (3) neutral polar amino acids; and (4) neutral non-polar amino acids. Representative amino acids within these various groups include, but are not limited to: (1) acidic (negatively charged) amino acids such as aspartic acid and glutamic acid; (2) basic (positively charged) amino acids such as arginine, histidine, and lysine; (3) neutral polar amino acids such as glycine, serine, threonine, cysteine, cystine, tyrosine, asparagine, and glutamine; and (4) neutral non-polar amino acids such as alanine, leucine, isoleucine, valine, proline, phenylalanine, tryptophan, and methionine.

LP82 variants having biological activities, in vivo or in vitro, that are similar or identical to those described herein, for example, the ability to prevent fat accretion in response to diets rich in fat and/or protein are contemplated by the present invention. LP82 variants, while being functionally related, may include amino acid sequences that differ in one or more positions from the sequence as shown in SEQ ID NO:2 or fragments thereof. LP82 variants that are useful in the methods of the present invention can be generated by deletion, insertion, inversion, and/or substitution of one or more amino acid residues in said LP82. LP82 variants can generally be made by solid phase or recombinant techniques in which, for example, single or multiple conservative amino acid substitutions are made, according to Table 1. Generally, in the case of multiple substitutions, it is preferred that between 80% to 99% of the residues of a LP82 variant are identical to the corresponding contiguous sequence as shown in SEQ ID NO:2; it is more preferable that between 90% to 99% of the residues of a LP82 variant are identical to the corresponding contiguous sequence as shown in SEQ ID NO:2; most preferably between 95% to 99% of the residues of a LP82 variant are identical to the corresponding contiguous sequence as shown in SEQ ID NO:2. Examples of most preferred LP82 variants include the LP82 variants as shown in SEQ ID NO:3.

Another class of LP82 variant that would be useful in the methods of the present invention is a LP82 polypeptide as defined herein further comprising one or more amino acid substitutions that result in an altered glycosylation pattern as compared to the corresponding unsubstituted LP82 polypeptide.

The term “N-glycosyled polypeptide” refers to polypeptides having one or more NXS/T motifs in which the nitrogen atom in the side chain amide of the asparagine is covalently bonded to a glycosyl group. “X” refers to any naturally occurring amino acid residue except proline. The “naturally occurring amino acids” are glycine, alanine, valine, leucine, isoleucine, proline, serine, threonine, cysteine, methionine, lysine, arganine, glutamic acid, asparatic acid, glutamine, asparagine, phenylalanine, histidine, tyrosine and tryptophan. N-glycosylated proteins are optionally O-glycosylation.

The term “O-glycosylated polypeptide” refers to polypeptides having one or more serines and/or threonine in which the oxygen atom in the side chain is covalently bonded to a glycosyl group. O-Glycosylated proteins are optionally N-glycosylation. Glycosylated polypeptides can be prepared recombinantly by expressing a gene encoding a polypeptide in a suitable mammalian host cell, resulting in glycosylation of side chain amides found in accessible NXT/S motifs on the polypeptide surface and/or of side chain alcohols of surface accessible serines and threonines. Specific procedures for recombinantly expressing genes in mammalian cells are provided hereinbelow. Other procedures for preparing glycosylated proteins are disclosed in EP 640,619 to Elliot and Burn. Unglycosylated polypeptides can be prepared recombinantly by expressing a gene encoding a polypeptide in a suitable prokaryotic host cell. The LP82 polypeptides and LP82 fragments and/or LP82 variants of the present invention can also be glycosylated or unglycosylated. A glycosylated polypeptide is modified with one or more monosaccharides or oligosaccharides. A monosaccharide is a chiral polyhydroxyalkanol or polyhydroxyalkanone which typically exists in hemiacetal form. An “oligosaccharide” is a polymer of from about 2 to about 18 monosaccharides which are generally linked by acetal bonds. One type of glycosyl group commonly found in glycosylated proteins is N-acetylneuraminic acid. A glycosylated polypeptide can be N-glycosylated and/or O-glycosylated, preferably N-glycosylated.

Another class of LP82 variant that may be useful in the methods of the present invention is a LP82 polypeptide as defined herein further comprising at least one oligopeptide or amino acid added onto the N-terminus and/or C-terminus. An “oligopeptide” is a chain of from two to about twenty-five amino acids connected at their N- and C-termini by peptide bonds. Suitable oligopeptides and amino acids are those that do not significantly decrease the biological activity of the LP82 polypeptide as defined herein and do not substantially detract from the desired pharmaceutical and pharmacological properties of the LP82 polypeptide. A preferred example of such a modification includes a LP82 polypeptide as defined herein further comprising a leader sequences found in other polypeptides, such as pretrypsinogen leader sequence.

The LP82 variants useful in the methods of the present invention also include LP82 polypeptides as defined herein further comprising one or more polyethylene glycol groups (hereinafter “PEG” groups). The PEG groups can be bonded to the N-terminus or to amine groups or thiol groups in the amino acid side chain(s) of LP82 polypeptides and fragments thereof. Suitable PEG groups generally have a molecular weight between about 5000 and 40,000 atomic mass units. Procedures for preparing PEGylated polypeptides are disclosed in Mumtaz and Bachhawat, Indian Journal of Biochemistry and Biophysics 28:346 (1991) and Francis et al., International Journal of Hematology 68:1 (1998).

The LP82 polypeptides as defined herein can also be expressed and used in a modified form, such as a fusion protein or a “tagged” protein. LP82 fusion proteins represent a hybrid protein molecule not found in nature comprising a translational fusion or enzymatic fusion in which two or more different proteins, fragments, or variants thereof are covalently linked on a single polypeptide chain. Human serum albumin, the C-terminal domain of thrombopoietin, the C-terminal extension peptide of hCG, and/or a Fc fragment are examples of proteins which could be fused with LP82 polypeptides, LP82 fragments and/or LP82 variants for use in the present invention. As used herein, “Fc fragment” of an antibody has the meaning commonly given to the term in the field of immunology. Specifically, this term refers to an antibody fragment which binds complement and is obtained by removing the two antigen binding regions (the Fab Fragments) from the antibody. Thus, the Fc fragment is formed from approximately equal sized fragments from both heavy chains, which associate through non-covalent interactions and disulfide bonds. The Fc Fragment includes the hinge regions and extends through the CH² and CH³ domains to the C-terminus of the antibody. Procedures for preparing fusion proteins are disclosed in EP394,827, Tranecker et al., Nature 331:84 (1988) and Fares, et al., Proc. Natl. Acad. Sci. USA 89:4304 (1992).

Many fusion proteins can be secreted by virtue of heterologous secretion signals in regions that can be removed prior to final preparation of the polypeptide. Such methodologies are described in many standard laboratory manuals, such as Sambrook, supra, Chapters 17.29-17.42 and 18.1-18.74; Ausubel, supra, Chapters 16, 17 and 18.

In a preferred process for protein expression and subsequent purification, the LP82 gene can be modified at the 5′ end to incorporate several histidine residues at the amino terminus of the LP82 protein resulting from its expression. This “histidine tag” enables a single-step protein purification method referred to as “immobilized metal ion affinity chromatography” (IMAC), essentially as described in U.S. Pat. No. 4,569,794, which hereby is incorporated by reference. The IMAC method enables rapid isolation of substantially pure recombinant LP82 protein starting from a crude extract of cells that express a modified recombinant protein, as described above.

The LP82 polypeptides and/or LP82 fragments and/or LP82 variants useful in the present invention as well as the nucleic acids encoding them may also be defined with reference to a percent identity similarity to either SEQ ID NO:2 or SEQ ID NO:1. Sequence identity refers to a comparison between two molecules using standard algorithms well known in the art. Although any suitable sequence comparison algorithm can be used for this purpose, for illustration, this embodiment shall be described with reference to the well-known Smith-Waterman algorithm using SEQ ID NO:2 as the reference sequence to define percent identity to a comparator sequence. When sequence identity is used with reference to a polypeptide, either the entire polypeptide may be used in the comparison or instead only a defined sub-region thereof.

The choice of parameter values for matches, mismatches, and inserts or deletions is arbitrary. A preferred set of values for use with the Smith-Waterman algorithm is set forth in the “maximum similarity segments” approach, which uses values of 1 for a matched residue, and −⅓ for a mismatched residue (See Waterman, Bulletin of Mathematical Biology, 46, 473-500, 1984). Insertions and deletions (indels), x, are weighted as follows:

Xₖ=1+k/3 where k is the number of residues in a given insert or deletion. For example, a comparator sequence that has 20 substitutions and 3 insertions relative to the 250 residue reference protein sequence would have an identity of:

[(1×250)−(⅓×20)−(1+3/3)1/250=96% identical.

Since LP82 variants can be produced easily by conventional recombinant or solid phase synthetic techniques known in the art, the methods of the present invention contemplates the use of LP82 variant polynucleotides and polypeptides to the extent that such variants have at least 70% and 80% identity to a contiguous sequence of LP82 nucleotides or a LP82 polypeptide as shown in SEQ ID NO:1 or 2, respectively while retaining substantially similar activity as the corresponding LP82 polynucleotide or LP82 polypeptide or fragment thereof, they are included within the scope of the present invention.

Fragments of the LP82 polypeptides, as well as variants thereof, may be generated by any number of suitable techniques, including chemical synthesis of any portion of SEQ ID NO:2., proteolytic digestion of LP82 polypeptides, or most preferably, by recombinant DNA mutagenesis techniques well known to the skilled artisan. See. e.g. K. Struhl, “Reverse biochemistry: Methods and applications for synthesizing yeast proteins in vitro,” Meth. Enzymol. 194, 520-535. For example, in a preferred method, a nested set of deletion mutations are introduced into a nucleic acid sequence encoding LP82 such that varying amounts of the protein coding region are deleted, either from the amino terminal end or from the carboxyl end of the protein molecule. This method can also be used to create internal fragments of the intact protein in which both the carboxyl and amino terminal ends are removed. Several appropriate nucleases can be used to create such deletions, for example Bal 31, or in the case of a single stranded nucleic acid molecule, mung bean nuclease. For simplicity, it is preferred that the LP82 gene be cloned into a single-stranded cloning vector, such as bacteriophage M13, or equivalent. If desired, the resulting gene deletion fragments can be subcloned into any suitable vector for propagation and expression of said fragments in any suitable host cell.

Functional fragments of the proteins disclosed herein may be produced as described above, preferably using cloning techniques to engineer smaller versions of the intact gene, lacking sequence from the 5′ end, the 3′ end, from both ends, or from an internal site. Fragments may be tested for biological activity using any suitable assay, for example, the ability of a protein fragment to induce mitogenesis, in vivo or in vitro.

Those skilled in the art will recognize that the LP82 gene could be obtained by a plurality of recombinant DNA techniques including, for example, hybridization, polymerase chain reaction (PCR) amplification, or de novo DNA synthesis. (See e.g., T. Maniatis et al. Molecular Cloning: A Laboratory Manual, 2d Ed. Chap. 14 (1989)).

Methods for constructing cDNA libraries in a suitable vector such as a plasmid or phage for propagation in prokaryotic or eukaryotic cells are well known to those skilled in the art. [See e.g. Maniatis et al. Supra]. Suitable cloning vectors are well known and are widely available.

The LP82 gene, or any fragment thereof, can be isolated from a tissue in which said gene is expressed, for example, placenta. In one method, mRNA is isolated, and first strand cDNA synthesis is carried out. A second round of DNA synthesis can be carried out for the production of the second strand. If desired, the double-stranded cDNA can be cloned into any suitable vector, for example, a plasmid, thereby forming a cDNA library oligonucleotide primers targeted to any suitable region of SEQ ID NO:1 can be used for PCR amplification of LP82. See e.g. PCR Protocols: A Guide to Method and Application, Ed. M. Innis et al., Academic Press (1990). The PCR amplification comprises template DNA, suitable enzymes, primers, and buffers, and is conveniently carried out in a DNA Thermal Cycler (Perkin Elmer Cetus, Norwalk, Conn.). A positive result is determined by detecting an appropriately-sized DNA fragment following agarose gel electrophoresis.

Skilled artisans will recognize that the proteins used in the present invention can be synthesized by a number of different methods, such as chemical methods well known in the art, including solid phase peptide synthesis or recombinant methods. Both methods are described in U.S. Pat. No. 4,617,149.

The principles of solid phase chemical synthesis of polypeptides are well known in the art and may be found in general texts in the area. See, e.g., H. Dugas and C. Penney, Bioorganic Chemistry (1981) Springer-Verlag, New York, 54-92. For example, peptides may be synthesized by solid-phase methodology utilizing an Applied Biosystems 430A peptide synthesizer (Applied Biosystems, Foster City, Calif.) and synthesis cycles supplied by Applied Biosystems. Additionally, peptides may be chemically ligated together by one skilled in the art of synthetic peptide synthesis.

The proteins used in the present invention can also be produced by recombinant DNA methods using the cloned LP82 gene. Recombinant methods are preferred if a high yield is desired. Expression of the cloned gene can be carried out in a variety of suitable host cells, well known to those skilled in the art. For this purpose, the LP82 gene is introduced into a host cell by any suitable means, well known to those skilled in the art. While chromosomal integration of the cloned gene is within the scope of the present invention, it is preferred that the gene be cloned into a suitable extra-chromosomally maintained expression vector so that the coding region of the LP82 gene is operably-linked to a constitutive or inducible promoter.

The basic steps in the recombinant production of LP82 protein or fragment or variant thereof are:

a) constructing a natural, synthetic or semi-synthetic DNA encoding said LP82 protein, fragment thereof, or variant thereof;

b) integrating said DNA into an expression vector in a manner suitable for expressing the LP82 protein or fragment or variant thereof, either alone or as a fusion protein;

c) transforming or otherwise introducing said vector into an appropriate eukaryotic or prokaryotic host cell forming a recombinant host cell,

d) culturing said recombinant host cell in a manner to express the LP82 protein or fragment and/or variant thereof; and

e) recovering and substantially purifying the LP82 protein or fragment and/or variant thereof by any suitable means, well known to those skilled in the art.

Prokaryotes may be employed in the production of recombinant LP82 polypeptides or fragments and/or variants thereof. For example, the E. coli K12 strain 294 (ATCC No. 31446) is particularly useful for the prokaryotic expression of foreign proteins. Other strains of E. coli, bacilli such as Bacillus subtilis, enterobacteriaceae such as Salmonella typhimurium or Serratia marcescans, various Pseudomonas species and other bacteria, such as Streptomyces, may also be employed as host cells in the cloning and expression of the recombinant proteins of this invention.

Promoter sequences suitable for driving the expression of genes in prokaryotes include β-lactamase [e.g. vector pGX2907, ATCC 39344, contains a replicon and β-lactamase gene], lactose systems [Chang et al., Nature (London), 275:615 (1978); Goeddel et al., Nature (London), 281:544 (1979)], alkaline phosphatase, and the tryptophan (trp) promoter system [vector pATH1 (ATCC 37695)], which is designed to facilitate expression of an open reading frame as a trpE fusion protein under the control of the trp promoter. Hybrid promoters such as the tac promoter (isolatable from plasmid pDR540, ATCC-3728′) are also suitable. Still other bacterial promoters, whose nucleotide sequences are generally known, may be ligated to DNA encoding the protein of the invention, using linkers or adapters to supply any required restriction sites. Promoters for use in bacterial systems also will contain a Shine-Dalgarno sequence operably-linked to the DNA encoding the desired polypeptides. These examples are illustrative rather than limiting.

The skilled artisan understands that choosing the most appropriate cloning vector or expression vector depends upon a number of factors including the availability of restriction enzyme sites, the type of host cell into which the vector is to be transfected or transformed, the purpose of the transfection or transformation (e.g., stable transformation as an extrachromosomal element, or integration into the host chromosome), the presence or absence of readily assayable or selectable markers (e.g., antibiotic resistance and metabolic markers of one type and another), and the number of copies of the gene desired in the host cell.

Vectors suitable to carry the nucleic acids of the present invention comprise RNA viruses, DNA viruses, lytic bacteriophages, lysogenic bacteriophages, stable bacteriophages, plasmids, viroids, and the like. The most preferred vectors are plasmids.

When preparing an expression vector the skilled artisan understands that there are many variables to be considered, for example, whether to use a constitutive or inducible promoter. The practitioner also understands that the amount of nucleic acid or protein to be produced dictates, in part, the selection of the expression system. Regarding promoter sequences, inducible promoters are preferred because they enable high level, regulatable expression of an operably-linked gene. The skilled artisan will recognize a number of suitable promoters that respond to a variety of inducers, for example, carbon source, metal ions, and heat. Other relevant considerations regarding an expression vector include whether to include sequences for directing the localization of a recombinant protein. For example, a sequence encoding a signal peptide preceding the coding region of a gene is useful for directing the extra-cellular export of a resulting polypeptide.

A suitable host cell is any eukaryotic cell which can accomodate high level expression of an exogenously introduced gene or protein and which will secrete said protein. Transformed host cells may be cultured under conditions well known to skilled artisans such that a LP82 polypeptide, LP82 fragment, and/or LP82 variant is expressed.

The polypeptides used in the methods of the present invention may be synthesized either by direct expression or as a fusion protein comprising the protein of interest as a translational fusion with another protein or peptide which may be removable by enzymatic or chemical cleavage. It is often observed in the production of certain peptides in recombinant systems that expression as a fusion protein prolongs the life span, increases the yield of the desired peptide, or provides a convenient means of purifying the protein. This is particularly relevant when expressing mammalian proteins in prokaryotic hosts. A variety of peptidases (e.g. enterokinase and thrombin) which cleave a polypeptide at specific sites or digest the peptides from the amino- or carboxy-termini (e.g. diaminopeptidase) of the peptide chain are known. Furthermore, particular chemicals (e.g. cyanogen bromide) will cleave a polypeptide chain at specific sites. The skilled artisan will appreciate the modifications necessary to the amino acid sequence (and synthetic or semi-synthetic coding sequence if recombinant means are employed) to incorporate site-specific internal cleavage sites (See e.g., P. Carter, “Site Specific Proteolysis of Fusion Proteins”, Chapter 13, in Protein Purification: From Molecular Mechanisms to Large Scale Processes, American Chemical Society, Washington, D.C. (1990)).

In addition to prokaryotes, a variety of amphibian expression systems such as frog oocytes, and mammalian cell systems can be used. The choice of a particular host cell depends to some extent on the particular expression vector used. Exemplary mammalian host cells suitable for use in the present invention include HepG-2 (ATCC HB 8065), CV-1 (ATCC CCL 70), LC-MK2 (ATCC CCL 7.1), 3T3 (ATCC CCL 92), CHO-KL (ATCC CCL 61), HeLa (ATCC CCL 2), RPMI8226 (ATCC CCL 155), H41IEC3 (ATCC CCL 1600), C127I (ATCC CCL 1616), HS-Sultan (ATCC CCL 1484), and BHK-21 (ATCC CCL 10), for example.

A wide variety of vectors are suitable for transforming mammalian host cells. For example, the pSV2-type vectors comprise segments of the simian virus 40 (SV40) genome required for transcription and polyadenylation. A large number of plasmid pSV2-type vectors have been constructed, such as pSV2-gpt, pSV2-neo, pSV2-dhfr, pSV2-hyg, and pSV2-b-globin, in which the SV40 promoter drives transcription of an inserted gene. These vectors are widely available from sources such as the American Type Culture Collection (ATCC), Rockville, Md., or the Northern Regional Research Laboratory (NRRL) Peoria, Ill.

Promoters suitable for expression in mammalian cells include the SV40 late promoter, promoters from eukaryotic genes, such as, for example, the estrogen-inducible chicken ovalbumin gene, the interferon genes, the glucocorticoid-inducible tyrosine aminotransferase gene, the thymidine kinase gene promoter, and the promoters of the major early and late adenovirus genes and the cytomegalovirus promoter.

Plasmid pRSVcat (ATCC 37152) comprises portions of a long terminal repeat of the Rous Sarcoma virus, a virus known to infect chickens and other host cells. This long terminal repeat contains a promoter which is suitable for use in the vectors of this invention. H. Gorman et al., Proc. Nat. Acad. Sci. (USA), 79, 6777 (1982). The plasmid pMSVi (NRRL B-15929) comprises the long terminal repeats of the Murine Sarcoma virus, a virus known to infect mouse and other host cells. The mouse metallothionein promoter has also been well characterized for use in eukaryotic host cells and is suitable for use in the present invention. This promoter is present in the plasmid pdBPV-MMTneo (ATCC 37224) which can serve as the starting material for the construction of other plasmids of the present invention.

Transfection of mammalian cells with vectors can be performed by a plurality of well-known processes including, protoplast fusion, calcium phosphate co-precipitation, electroporation and the like.

Some viruses also make appropriate vectors. Examples include the adenoviruses, the adeno-associated viruses, the vaccinia virus, the herpes viruses, the baculoviruses, and the rous sarcoma virus, as described in U.S. Pat. No. 4,775,624.

Eukaryotic microorganisms such as yeast and other fungi are also suitable host cells. The yeast Saccharomyces cerevisiae is the preferred eukaryotic microorganism. Other yeasts such as Kluyveromyces lactis and Pichia pastoris are also suitable. For expression in Saccharomyces, the plasmid YRp7 (ATCC-40053), for example, may be used. See, e.g., L. Stinchcomb et al., Nature, 282, 39 (1979); J. Kingsman et al., Gene, 7, 141 (1979); S. Tschemper et al., Gene, 10, 157 (1980). Plasmid YRp7 contains the TRP1 gene which provides a selectable marker for use in a trp1 auxotrophic mutant.

An expression vector carrying the cloned LP82 gene or fragment thereof is transformed or transfected into a suitable host cell using standard methods. Cells that contain the vector are propagated under conditions suitable for expression of the recombinant LP82 protein. For example, if the recombinant gene has been placed under the control of an inducible promoter, suitable growth conditions would incorporate the appropriate inducer. The recombinantly-produced protein may be purified from cellular extracts of transformed cells by any suitable means.

The percent identity between nucleic acids or proteins is determined by any suitable comparison algorithm, well known to the skilled artisan. By percent identity is meant the number of residues that are identical between optimally aligned nucleic acids (or proteins), divided by the total length (including gaps) of the shortest sequence of the pair or group being compared. Nucleic acid or protein sequences are optimally aligned to achieve the greatest degree of similarity, allowing for gaps, using any suitable algorithm, for example, a dynamic programming algorithm (See e.g. Smith and Waterman, J. Mol. Biol. 147, 195 (1985), BLASTA (Altschul et al. J. Mol. Biol. 215, 403 (1990); or FASTA (Lipman & Pearson, Science, 227, 1435 (1985), herein incorporated by reference. Such alignments are carried out with the paramters set to maximize the alignment score obtained for a pair of sequences being compared.

The LP82 cDNA (viz. SEQ ID NO:1) and related nucleic acid molecules that encode SEQ ID NO:2, 4, or 6, or fragments and/or variants thereof, may be produced by chemical synthetic methods. The synthesis of nucleic acids is well known in the art. See, e.g., E L. Brown, R. Belagaje, M J. Ryan, and H G. Khorana, Methods in Enzymology, 68:109-151 (1979). Fragments of the DNA sequence corresponding to the LP82 gene may be generated using a conventional DNA synthesizing apparatus using phosphoramidite chemistry, thereafter ligating the fragments so as to reconstitute the entire gene. Alternatively, phosphotriester chemistry may be employed to synthesize the nucleic acids of this invention. (See, e.g., M J. Gait, ed., Oligonucleotide Synthesis, A Practical Approach, (1984)).

In an alternative methodology, namely PCR, the DNA sequences disclosed and described herein, comprising, for example, a portion or all of SEQ ID NO:1 can be produced from a plurality of starting materials. For example, starting with a cDNA preparation (e.g. cDNA library) derived from a tissue that expresses the LP82 gene, suitable oligonucleotide primers complementary to SEQ ID NO:1 or to any sub-region therein, are prepared as described in U.S. Pat. No. 4,889,818, hereby incorporated by reference. Other suitable protocols for the PCR are disclosed in PCR Protocols: A Guide to Method and Applications, Ed. Michael A. Innis et al., Academic Press, Inc. (1990). Using PCR, any region of the LP82 gene can be targeted for amplification such that full or partial length gene sequences may be produced.

The methods of the present invention may also utilize LP82 antibodies. Exemplary antibodies include polyclonal, monoclonal, humanized, bispecific, and heteroconjugate antibodies.

The LP82 antibodies used in the present invention may include polyclonal antibodies. Methods of preparing polyclonal antibodies are known to the skilled artisan. Polyclonal antibodies can be raised in a mammal, for example, by one or more injections of an immunizing agent and, if desired, an adjuvant. Typically, the immunizing agent and/or adjuvant will be injected in the mammal by multiple subcutaneous or intraperitoneal injections. The immunizing agent may include the LP82 polypeptide or a fusion protein thereof. It may be useful to conjugate the immunizing agent to a protein known to be immunogenic in the mammal being immunized. Examples of such immunogenic proteins include, but are not limited to, keyhole limpet hemocyanin, serum albumin, bovine thyroglobulin, and soybean trypsin inhibitor. Examples of adjuvants which may be employed include Freund's complete adjuvant and MPL-TDM adjuvant (monophosphoryl Lipid A, synthetic trehalose dicorynomycolate). The immunization protocol may be selected by one skilled in the art without undue experimentation.

LP82 antibodies may, alternatively, be monoclonal antibodies. Monoclonal antibodies may be prepared using hybridoma methods, such as those described by Kohler and Milstein, Nature 256(5517): 495-7 (1975). In a hybridoma method, a mouse, hamster, or other appropriate host animal is typically immunized with an immunizing agent to elicit lymphocytes that produce or are capable of producing antibodies that will specifically bind to the immunizing agent. Alternatively, the lymphocytes may be immunized in vitro.

The immunizing agent will typically include an LP82 polypeptide or a fusion protein thereof. Generally, either peripheral blood lymphocytes (“PBLs”) are used, if cells of human origin are desired, or spleen cells or lymph node cells are used, if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986) pp. 59-103]. Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which prevents the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif., and the American Type Culture Collection, Rockville, Md. Human myeloma and mouse-human heteromyeloma cell lines also have been described for the production of human monoclonal antibodies [Kozbor, J. Immunol. 133(6): 3001-5 (1984); Brodeur, et al., Monoclonal Antibody Production Techniques and Applications, Marcel Dekker, Inc., NY (1987) pp. 51-63].

The culture medium in which the hybridoma cells are cultured can then be assayed for the presence of monoclonal antibodies directed against an LP82 polypeptide. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Rodbard, Anal. Biochem. 107(1): 220-39 (1980).

After the desired hybridoma cells are identified, the clones may be subcloned by limiting dilution procedures and grown by standard methods [Goding, Monoclonal Antibodies: Principles and Practice, Academic Press, (1986) pp. 59-103]. Suitable culture media for this purpose include, for example, Dulbecco's Modified Eagle's Medium and RPMI-1640 medium. Alternatively, the hybridoma cells may be grown in vivo as ascites in a mammal.

Monoclonal antibodies may also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA may be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences [U.S. Pat. No. 4,816,567; Morrison, et al., Proc. Natl. Acad. Sci. USA 81(21):6851-5 (1984)] or by covalently joining to the immunoglobulin coding sequence all or part of the coding sequence for a non-immunoglobulin polypeptide. Such a non-immunoglobulin polypeptide can be substituted for the constant domains of an antibody of the invention or can be substituted for the variable domains of one antigen-combining site of an antibody of the invention to create a chimeric bivalent antibody.

LP82antibodies may be monovalent antibodies. Methods for preparing monovalent antibodies are well known in the art. For example, one method involves recombinant expression of immunoglobulin light chain and modified heavy chain. The heavy chain is truncated generally at any point in the Fc region so as to prevent heavy chain crosslinking. Alternatively, the relevant cysteine residues are substituted with another amino acid residue or are deleted so as to prevent crosslinking.

In vitro methods are also suitable for preparing monovalent antibodies. Digestion of antibodies to produce fragments thereof, particularly Fab fragments, can be accomplished using routine techniques known in the art.

The LP82 antibodies used in the methods of the invention may further comprise humanized antibodies or human antibodies. Humanized forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include human immunoglobulins (recipient antibody) in which residues from a complementary-determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin, and all or substantially all of the FR regions are those of a human immunoglobulin consensus sequence. The humanized antibody optimally also will comprise at least a portion of an immunoglobulin constant region (Fc), typically that of a human immunoglobulin [Jones, et al., Nature 321(6069): 522-5 (1986); Riechmann, et al., Nature 332(6162): 323-7 (1988); and Presta, Curr. Op. Struct. Biol. 2: 593-6 (1992)].

Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers [Jones, et al., Nature 321(6069):522-5 (1986); Riechmann, et al., Nature 332(6162):323-7 (1988); Verhoeyen, et al., Science 239(4847):1534-6 (1988)), by substituting rodent CDRs or CDR sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human LP82 antibodies can also be produced using various techniques known in the art, including phage display libraries [Hoogenboom and Winter, J. Mol. Biol. 227:381-8 (1992); Marks, et al., J. Mol. Biol. 222:581-97 (1991)]. The techniques of Cole, et al., and Boerner, et al., are also available for the preparation of human monoclonal antibodies (Cole, et al., Monoclonal Antibodies and Cancer Therapy, Alan R. Liss, p. 77 (1985) and Boerner, et al., J. Immunol. 147:86-95 (1991)]. Similarly, human LP82 antibodies can be made by introducing human immunoglobulin loci into transgenic animals, e.g., mice in which the endogenous immunoglobulin genes have been partially or complete inactivated. Upon challenge, human LP82 polypeptide antibody production is observed, which closely resembles that seen in humans in all respects, including gene rearrangement, assembly and antibody repertoire. This approach is described, for example, in U.S. Pat. Nos. 5,545,807; 5,545,806; 5,569,825; 5,625,126; 5,633,425; 5,661,016.

Bispecific antibodies are monoclonal, preferably human or humanized, antibodies that have binding specificities for at least two different antigens. In the present case, one of the binding specificities may be for an LP82 polypeptide, the other one is for any other antigen, and preferably for a cell-surface protein or receptor or receptor subunit. Methods for making bispecific antibodies are known in the art. Antibodies with more than two valencies are contemplated. For example, trispecific antibodies can be prepared [Tutt, et al., J. Immunol. 147:60-9 (1991)].

The use of heteroconjugated antibodies is also contemplated as part of the present invention. Heteroconjugate antibodies are composed of two covalently joined antibodies. Such antibodies have, for example, been proposed to target immune system cells to unwanted cells [U.S. Pat. No. 4,676,980], and for treatment of HIV infection [WO 91/00360; WO 92/20373). It is contemplated that the antibodies may be prepared in vitro using known methods in synthetic protein chemistry, including those involving crosslinking agents. For example, immunotoxins may be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

The usefulness of a LP82 agonist, LP82 antagonist, LP82 polynucleotide, LP82 polypeptide, LP82 variant, and/or LP82 antibody for the methods of the present invention can be determined by one skilled in the art without undue experimentation by application of the methods or assays described herein or otherwise known in the art. A LP82 agonist, LP82 antagonist, LP82 polynucleotide, LP82 polypeptide, LP82 variant, and/or LP82 antibody of the present invention can easily be tested for biological activity or functionality as described herein or as otherwise known in the art. The biological activity or functionality of a LP82 agonist, LP82 antagonist, LP82 polynucleotide, LP82 polypeptide, LP82 variant, and/or LP82 antibody can be assessed or quantified using either in vitro models or in vivo models of obesity, diabetes, body composition, or other metabolic disorders as described herein (see Examples) or assays otherwise known in the art.

The present invention still further relates to methods for identifying compounds which modulate the expression of the mammalian LP82 gene and/or the synthesis or activity of mammalian LP82 gene products. Such compounds include therapeutic compounds which can be used as pharmaceutical compositions to reduce or eliminate the symptoms of mammalian body weight disorders such as obesity, cachexia, and anorexia. Cellular and non-cellular assays are described that can be used to identify compounds that interact with the LP82 gene and/or gene product, e.g., modulate the activity of the LP82 gene and/or bind to the LP82 gene product. Such cell-based assays of the invention utilize cells, cell lines, or engineered cells or cell lines that express the LP82 gene product.

First, cell-based systems can be used to identify compounds that may act to ameliorate body weight disorder symptoms. Such cell systems can include, for example, recombinant or non-recombinant cell, such as cell lines, that express the LP82 gene. In utilizing such cell systems, cells that express LP82 may be exposed to a compound suspected of exhibiting an ability to ameliorate body weight disorder symptoms, at a sufficient concentration and for a sufficient time to elicit such an amelioration of such symptoms in the exposed cells.

After exposure, the cells can be assayed to measure alterations in the expression of the LP82 gene, e.g., by assaying cell lysates for LP82 mRNA transcripts (e.g., by Northern analysis) or for LP82 gene products expressed by the cell; compounds that modulate expression of the LP82 gene are good candidates as therapeutics.

In addition, animal-based systems or models for a mammalian body weight disorder, for example, transgenic mice containing a human or altered form of LP82 gene, may be used to identify compounds capable of ameliorating symptoms of the disorder. Such animal models may be used as test substrates for the identification of drugs, pharmaceuticals, therapies and interventions. For example, animal models may be exposed to a compound suspected of exhibiting an ability to ameliorate symptoms, at a sufficient concentration and for a sufficient time to elicit such an amelioration of body weight disorder symptoms. The response of the animals to the exposure may be monitored by assessing the reversal of the symptoms of the disorder.

With regard to intervention, any treatments that reverse any aspect of body weight disorder-like symptoms should be considered as candidates for human therapeutic intervention in such a disorder. Dosages of test agents may be determined by deriving dose-response curves

In one embodiment, such methods comprise contacting a compound to a cell that expresses a LP82 gene, measuring the level of LP82 gene expression, gene product expression, or gene product activity, and comparing this level to the level of LP82 gene expression, gene product expression, or gene product activity produced by the cell in the absence of the compound, such that if the level obtained in the presence of the compound differs from that obtained in its absence, a compound that modulates the expression of the mammalian LP82 gene and/or the synthesis or activity of mammalian LP82 gene products has been identified.

In an alternative embodiment, such methods comprise administering a compound to a host, e.g., a transgenic animal that expresses a LP82 transgene or a LP82 variant transgene, and measuring the level of LP82 gene expression, gene product expression, or gene product activity. The measured level is compared to the level of LP82 gene expression, gene product expression, or gene product activity in a host that is not exposed to the compound, such that if the level obtained when the host is exposed to the compound differs from that obtained when the host is not exposed to the compound, a compound that modulates the expression of the mammalian LP82 gene and/or the synthesis or activity of mammalian LP82 gene products, and/or the symptoms of a mammalian body weight disorder, such as obesity, cachexia, or anorexia, has been identified.

Methods of the present invention can comprise, for example administering compounds which modulate the expression of a mammalian LP82 gene and/or the synthesis or activity of a mammalian LP82 gene product, so that symptoms of the body weight disorder are ameliorated. Alternatively, in those instances whereby the mammalian body weight disorder results from LP82 gene mutations, such methods can comprise supplying the mammal with a nucleic acid molecule encoding an unimpaired LP82 gene product such that an unimpaired LP82 gene product is expressed and symptoms of the disorder are ameliorated.

In another embodiment of methods for the treatment of mammalian body weight disorders resulting from LP82 gene mutations, such methods can comprise supplying the mammal with a cell comprising a nucleic acid molecule that encodes an unimpaired LP82 gene product such that the cell expresses the unimpaired LP82 gene product, and symptoms of the disorder are ameliorated.

Alternatively, symptoms of certain body weight disorders such as, for example, cachexia and anorexia, which involve a lower than normal body weight phenotype, may be ameliorated by inhibiting the level of LP82 gene expression and/or LP82 gene product activity. Methods for inhibiting or reducing the level of LP82 gene product synthesis or expression can include, for example, methods such as inhibitory antisense, ribozyme and triple helix approaches. In another embodiment, symptoms of body weight disorders may be ameliorated by decreasing the level of LP82 gene expression and/or LP82 gene product activity by using LP82 gene sequences in conjunction with well-known antisense, gene knock-out, ribozyme and/or triple helix methods to decrease the level of LP82 gene expression. Among the compounds that may exhibit the ability to modulate the activity, expression or synthesis of the LP82 gene, including the ability to ameliorate the symptoms of a mammalian body weight disorder, are antisense, ribozyme, and triple helix molecules. Such molecules may be designed to reduce or inhibit either unimpaired, or if appropriate, mutant target gene activity. Techniques for the production and use of such molecules are well known to those of skill in the art.

Antisense RNA and DNA molecules act to directly block the translation of mRNA by hybridizing to targeted mRNA and preventing protein translation. Antisense approaches involve the design of oligonucleotides that are complementary to a target gene mRNA. The antisense oligonucleotides will bind to the complementary target gene mRNA transcripts and prevent translation. Absolute complementarily, although preferred, is not required. A sequence “complementary” to a portion of an RNA, as referred to herein, means a sequence having sufficient complementarily to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarily and the length of the antisense nucleic acid. Generally, the longer the hybridizing nucleic acid, the more base mismatches with an RNA it may contain and still form a stable duplex (or triplex, as the case may be) One skilled in the art can ascertain a tolerable degree of mismatch by use of standard procedures to determine the melting point of the hybridized complex.

In one embodiment, oligonucleotides complementary to non-coding regions of the LPS2 gene could be used in an antisense approach to inhibit translation of endogenous LP82 mRNA. Antisense nucleic acids should be at least six nucleotides in length, and are preferably oligonucleotides ranging from 6 to about 50 nucleotides in length. In specific aspects the oligonucleotide is at least nucleotides, at least 17 nucleotides, at least 25 nucleotides or at least 50 nuclectides.

Regardless of the choice of target sequence, it is preferred that in vitro studies are first performed to quantitate the ability of the antisense oligonucleotide to inhibit gene expression. It is preferred that these studies utilize controls that distinguish between antisense gene inhibition and nonspecific biological effects of oligonucleotides. It is also preferred that these studies compare levels of the target RNA or protein with that of an internal control RNA or protein. Additionally, it is envisioned that results obtained using the antisense oligonucleotide are compared with those obtained using a control oligonucleotide. It is preferred that the control oligonucleotide is of approximately the same length as the test oligonucleotide and that the nucleotide sequence of the oligonucleotide differs from the antisense sequence no more than is necessary to prevent specific hybridization to the target sequence. The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors in vivo), or agents facilitating transport across the cell membrane (see, e.g., Letsinger, et al., 1989, Proc. Natl. Acad. Sci. U.S.A. 86:6553-6556; Lemaitre, et al., 1987, Proc. Natl. Acad. Sci. U.S.A. 84:648-652; PCT Publication No. WO88/09810, published Dec. 15, 1988) or the blood-brain barrier (see, e.g., PCT Publication No. WO89/10134, published Apr. 25, 1988), hybridization-triggered cleavage agents (see, e.g., Krol et al., 1988, BioTechniques 6:958-976) or intercalating agents (see, e.g. Zon, 1988, Pharm. Res. 5:539-549). To this end, the oligonucleotide may be conjugated to another molecule, e.g., a peptide, hybridization triggered cross-linking agent, transport agent, hybridization-triggered cleavage agent, etc. The antisense oligonucleotide may comprise at least one modified base moiety which is selected from the group including, but not limited to, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D- is galactosylqueosine, inosine, NG-isopentenyladenine, 1-methylguanine, 1-methylinosine, 2,2-dimethyl guanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid-methylester, uracil-5-oxyacetic acid (v), 5-methyl-2-thiouracil, 3-(3-amino-3-N-2-carboxypropyl) uracil, (acp3)w, and 2,6-diaminopurine. The antisense oligonucleotide may also comprise at least one modified sugar moiety selected from the group including but not limited to arabinose, 2-fluoroarabinose, xylulose, and hexose.

In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet another embodiment, the antisense oligonucleotide is an a-anomeric oligonucieotide. An a-anomeric oligonucleotide forms specific double-stranded hybrids with complementary RNA in which, contrary to the usual, the strands run parallel to each other (Gautier, et al., 1987, Nucl. Acids Res. 15:6625-6641). The oligonucleotide is a 2′-O-methylribonucleotide (Inoue, et al., 1987, Nucl. Acids Res. 15:6131-6148), or a chimeric RNA-DNA analogue (Inoue, et al., 1987, FEBS Lett. 215:327-330). Oligonucleotides of the invention may he synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein, et al. (1988, Nucl. Acids Res. 16:3209), methylphosphonate oligonucleotides can be prepared by use of controlled pore glass polymer supports (Sarin, et al., 1988, Proc. Natl. Acad. Sci. U.S.A. 85:7448-7451), etc. While antisense nucleotides complementary to the target gene coding region sequence could be used, those complementary to the transcribed, untranslated region are most preferred.

Antisense molecules should be delivered to cells that express the target gene in vivo. A number of methods have been developed for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systemically. However, it is often difficult to achieve intracellular concentrations of the antisense sufficient to suppress translation of endogenous mRNAs. Therefore a preferred approach utilizes a recombinant DNA construct in which the antisense oligonucleotide is placed under the control of a strong pol III or pol II promoter. The use of such a construct to transfect target cells in the patient will result in the transcription of sufficient amounts of single stranded RNAs that will form complementary base pairs with the endogenous target gene transcripts and thereby prevent translation of the target gene mRNA. For example, a vector can be introduced e.g., such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art vectors can be plasmid, viral, or others known in the art, used for replication and expression in mammalian cells. Expression of the sequence encoding the antisense RNA can be by any promoter known in the art to act in mammalian, preferably human cells. Such promoters can be inducible or constitutive. Such promoters include but are not limited to: the SV40 early promoter region (Bernoist and Chambon, 1981, Nature 290:304-310), the promoter contained in the 3′ long terminal repeat of Rous sarcoma virus (Yamamoto, et al., 1980, Cell 22:787-797), the herpes thymidine kinase promoter (Wagner, et al., 1961, Proc. Natl. Acad. Sci. U.S.A. 78:1441-1445), the regulatory sequences of the metallothionein gene (Brinster, et al., 1982, Nature 296:39-42), etc. Any type of plasmid, cosmid, YAC or viral vector can be used to prepare the recombinant DNA construct which can be introduced directly into the tissue site. Alternatively, viral vectors can be used that selectively infect the desired tissue, in which case administration may be accomplished by another route (e.g., systemically). Ribozyme molecules designed to catalytically cleave target gene mRNA transcripts can also be used to prevent translation of target gene mRNA and, therefore, expression of target gene product. (See, e.g., PCT International Publication WO90/11364, published Oct. 4, 1990; Sarver, et al., 1990, Science 247:1222-122S).

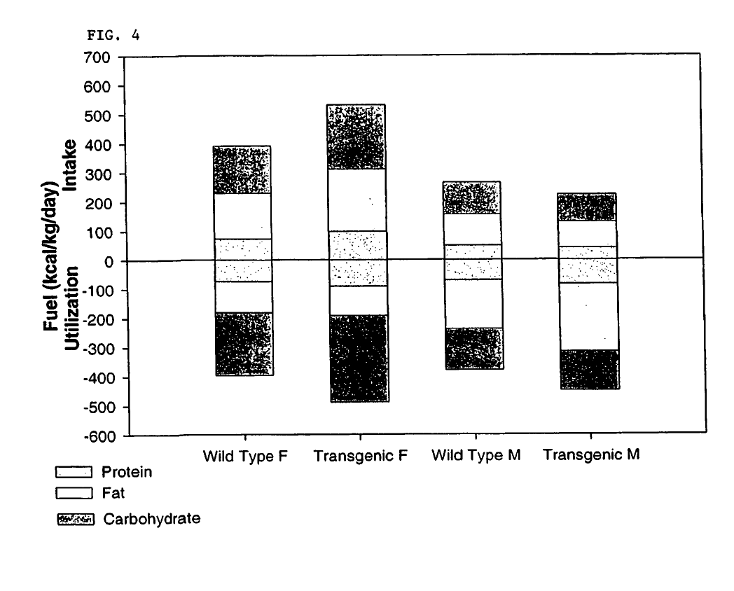

Ribozymes are enzymatic RNA molecules capable of catalyzing the specific cleavage of RNA. (For a review, see Rossi, 1994, Current Biology 4:469-471). The mechanism of ribozyme action involves sequence specific hybridization of the ribozyme molecule to complementary target RNA, followed by an endonucleolytic cleavage event. The composition of ribozyme molecules must include one or more sequences complementary to the target gene mRNA, and must include the well known catalytic sequence responsible for mRNA cleavage (For this sequence, see, e.g., U.S. Pat. No. 5,093,246, which is incorporated herein by reference in its entirety). While ribozymes that cleave mRNA at site specific recognition sequences can be used to destroy target gene mRNAs, the use of hammerhead ribozymes is preferred. Hammerhead ribozymes cleave mRNAs at locations dictated by 2S flanking regions that form complementary base pairs with the target mRNA. The sole requirement is that the target mRNA have the following sequence of two bases: 51-UG-31. The construction and production of hammerhead ribozymes is well known in the art and is described more fully in Myers, 1995, Molecular Biology and Biotechnology: A Comprehensive Desk Reference, VCH Publishers, New York, (see especially FIG. 4, page 833) and in Haseloff and Gerlach, 1988, Nature, 334, 585-591. Preferably the ribozyme is engineered so that the cleavage recognition site is located near the 51 end of the target gene mRNA, i.e., to increase efficiency and minimize the intracellular-accumulation of non-functional mRNA transcripts.

The ribozymes of the present invention also include RNA endoribonucl eases (hereinafter “Cech-type ribozymes”) such as the one that occurs naturally in Tetrahymena thermophila known as the IVS, or L-19 IVS RNA) and that has been extensively described by Thomas Cech and collaborators (Zaug, et al., 1984, Science, 224, 574-578; Zaug and Cech, 1986, Science, 231, 470-475; Zaug, et al., 1986, Nature, 324, 429-433; published international patent application No. WO 88/04300, Been and Cech, 1986, Cell, 47:207-216). The Cech-type ribozymes have an eight base pair active site which hybridizes to a target RNA sequence whereafter cleavage of the target RNA takes place.

The invention encompasses the use of those Cech-type ribozymes which target eight base-pair active site sequences that are present in the target gene. As in the antisense approach, the ribozymes can be composed of modified oligonucleotides (e.g., for improved stability, targeting, etc.) and should be delivered to cells that express the target gene in vivo. A preferred method of delivery involves using a DNA construct “encoding” the ribozyme under the control of a strong constitutive pol III or pol II promoter, so that transfected cells will produce sufficient quantities of the ribozyme to destroy endogenous target gene messages and inhibit translation. Because ribozymes unlike antisense molecules, are catalytic, a lower intracellular concentration is required for efficiency.

Endogenous target gene expression can also be reduced by inactivating or “knocking out” the target gene or its promoter using targeted homologous recombination (e.g., see Smithies, et al., 1985, Nature 317, 230-234; Thomas and Capecchi, 1987, Cell 51, 503-512; Thompson, et al., 1989, Cell 5, 313-321). For example, a mutant, non-functional target gene (or a completely unrelated DNA sequence) flanked by DNA homologous to the endogenous target gene (either the coding regions or regulatory regions of the target gene) can be used, with or without a selectable marker and/or a negative selectable marker, to transfect cells that express the target gene in vivo. Insertion of the DNA construct, via targeted homologous recombination, results in inactivation of the target gene. Such approaches are particularly suited in the agricultural field where modifications to ES (embryonic stem) cells can be used to generate animal offspring with an inactive target gene (e.g., see Thomas and Capecchi, 1987 and Thompson, 1989, supra) However this approach can be adapted for use in humans provided the recombinant DNA constructs are directly administered or targeted to the required site in vivo using appropriate viral vectors.

Alternatively, endogenous target gene expression can be reduced by targeting deoxyribonucleotide sequences complementary to the regulatory region of the target gene (i.e., the target gene promoter and/or enhancers) to form triple helical structures that prevent transcription of the target gene in target cells in the body. (See generally, Helene, 1991, Anticancer Drug Des., 6;569-584; Helene, et al., 1992, Ann. N.Y. Acad. Sci., 660:27-36; and Maher, 1992, Bioassays 14:807-815).

Nucleic acid molecules to be used in triplex helix formation for the inhibition of transcription should be single stranded and composed of deoxynucleotides. The base composition of these oligonucleotides must be designed to promote triple helix formation via Hoogsteen base pairing rules, which generally require sizeable stretches of either purines or pyrimidines to be present on one strand of a duplex. Nucleotide sequences may be pyrimidine-based, which will result in TAT and CGC triplets across the three associated strands of the resulting triple helix. The pyrimidine-rich molecules provide base complementarily to a purine-rich region of a single strand of the duplex in a parallel orientation to that strand. In addition, nucleic acid molecules may be chosen that are purine-rich, for example, contain a stretch of G residues. These molecules will form a triple helix with a DNA duplex that is rich in GC pairs, in which the majority of the purine residues are located on a single strand of the targeted duplex, resulting in GGC triplets across the three strands in the triplex. Alternatively, the potential sequences that can be targeted for triple helix formation may be increased by creating a so-called “switchback” nucleic acid molecule. Switchback molecules are synthesized in an alternating 51-31, 31-51 manner, such that they base pair with first one strand of a duplex and then the other, eliminating the necessity for a sizeable stretch of either purines or pyrimidines to be present on one strand of a duplex.

In instances wherein the antisense, ribozyme, and/or triple helix molecules described herein are utilized to inhibit mutant gene expression, it is possible that the technique may so efficiently reduce or inhibit the transcription (triple helix) and/or translation (antisense, ribozyme) of mRNA produced by normal target gene alleles that the possibility may arise wherein the concentration of normal target gene product present may be lower than is necessary for a normal phenotype. In such cases, to ensure that substantially normal levels of target gene activity are maintained, therefore, nucleic acid molecules that encode and express target gene polypeptides exhibiting normal target gene activity may, be introduced into cells via gene therapy methods such as those described, below, in Section 5.9.2 that do not contain sequences susceptible to whatever antisense, ribozyme, or triple helix treatments are being utilized. Alternatively, in instances whereby the target gene encodes an extracellular protein, it may be preferable to co-administer normal target gene protein in order to maintain the requisite level of target gene activity.

Anti-sense RNA and DNA, ribozyme, and triple helix molecules useful in the methods of the invention may be prepared by any method known in the art for the synthesis of DNA and RNA molecules, as discussed above. These include techniques for chemically synthesizing oligodeoxy-ribonucleotides and oligoribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

For therapeutic utility, an effective amount of LP82 polypeptide, LP82 variant, and/or LP82 antibody is administered to a mammal in need thereof in a dose between about 0.1 and 1000 μg/kg body weight. In practicing the methods contemplated by this invention, the LP82 polypeptides, LP82 variants, and/or LP82 antibodies as defined herein can be administered in multiple doses per day, in single daily doses, in weekly doses, or at any other regular interval. The amount per administration and frequency of administration will be determined by a physician and depend on such factors as the nature and severity of the disease, and the age and general health of the patient.

The present invention also provides a pharmaceutical composition comprising as the active agent an LP82 agonist, LP82 antagonist, LP82 polynucleotide, LP82 polypeptide, LP82 fragment, LP82 variant, and/or LP82 antibody, and/or a pharmaceutically acceptable non-toxic salt thereof, and a pharmaceutically acceptable solid or liquid carrier. For example, compounds comprising at least one LP82 agonist, LP82 antagonist, LP82 polynucleotide, LP82 polypeptide, LP82 fragment, LP82 variant, and/or LP82 antibody can be admixed with conventional pharmaceutical carriers and excipients, and used in the form of tablets, capsules, elixirs, suspensions, syrups, wafers, parenteral formulations, and the like. The compositions comprising at least one LP82 agonist, LP82 antagonist, LP82 polynucleotide, LP82 polypeptide, LP82 fragment, LP82 variant, and/or LP82 antibody, will contain from about 0.1% to 90% by weight of the active compound, and more generally from about 10% to 30%. The compositions may contain common carriers and excipients such as corn-starch or gelatin, lactose, sucrose, microcrystalline cellulose, kaolin, mannitol, dicalcium phosphate, sodium chloride, and alginic acid.

As a general proposition, the total pharmaceutically effective amount of a LP82 agonist, LP82 antagonist, LP82 polynucleotide, LP82 polypeptide, LP82 fragment, LP82 variant, and/or LP82 antibody administered parenterally per dose will be in the range of about 1 μg/kg/day to 10 mg/kg/day of patient body weight, particularly 2 mg/kg/day to 8 mg/kg/day, more particularly 2 mg/kg/day to 4 mg/kg/day, even more particularly 2.2 mg/kg/day to 3.3 mg/kg/day, and finally 2.5 mg/kg/day, although, as noted above, this will be subject to therapeutic discretion. More preferably, this dose is at least 0.01 mg/kg/day. If given continuously, a LP82 agonist, LP82 antagonist, LP82 polynucleotide, LP82 polypeptide, LP82 fragment, LP82 variant, and/or LP82 antibody is typically administered at a dose rate of about 1 μg/kg/hour to about 50 μg/kg/hour, either by 1-4 injections per day or by continuous subcutaneous infusions, for example, using a mini-pump. An intravenous bag solution may also be employed. The length of treatment needed to observe changes and the interval following treatment for responses to occur appears to vary depending on the desired effect.

Pharmaceutical compositions containing a LP82 agonist, LP82 antagonist, LP82 polynucleotide, LP82 polypeptide, LP82 fragment, LP82 variant, and/or LP82 antibody may be administered orally, rectally, intracranially, parenterally, intracisternally, intravaginally, intraperitoneally, topically (as by powders, ointments, drops or transdermal patch), transdermally, intrathecally, bucally, or as an oral or nasal spray. By “pharmaceutically acceptable carrier” is meant a non-toxic solid, semisolid or liquid filler, diluent, encapsulating material or formulation auxiliary of any type. The term “parenteral” as used herein includes, but is not limited to, modes of administration which include intravenous, intramuscular, intraperitoneal, intrasternal, subcutaneous and intra-articular injection, infusion and implants comprising a LP82 agonist, LP82 antagonist, LP82 polynucleotide, LP82 polypeptide, LP82 fragment, LP82 variant, and/or LP82 antibody.

The compounds can be formulated for oral or parenteral administration. A preferred parenteral formulation for subcutaneous administration would comprise a buffer (phosphate, citrate, acetate, borate, TRIS), salt (NaCl, KCl), divalent metal (Zn, Ca), and isotonicty agent (glycerol, mannitol), detergent (Polyoxyethylene sorbitan fatyy acid esters, poloxamer, ddicusate sodium, sodium lauryl sulfate), antioxidants (ascorbic acid), and antimicrobial agent (phenol, m-cresol, alcohol, benzyl alcohol, butylparben, methylparaben, ethylparaben, chlorocresol, phenoxyethanol, phenylethyl alcohol, propylparaben.

For intravenous (IV) use, a LP82 agonist, LP82 antagonist, LP82 polynucleotide, LP82 polypeptide, LP82 fragment, LP82 variant, and/or LP82 antibody is administered in commonly used intravenous fluid(s) and administered by infusion. Such fluids, for example, physiological saline, Ringers solution or 5% dextrose solution can be used.

For intramuscular preparations, a sterile formulation, preferably a suitable soluble salt form of a LP82 agonist, LP82 antagonist, LP82 polynucleotide, LP82 polypeptide, LP82 fragment, LP82 variant, and/or LP82 antibody such as the hydrochloride salt, can be dissolved and administered in a pharmaceutical diluent such as pyrogen-free water (distilled), physiological saline or 5% glucose solution. A suitable insoluble form of the compound may be prepared and administered as a suspension in an aqueous base or a pharmaceutically acceptable oil base, e.g. an ester of a long chain fatty acid such as ethyl oleate.

A LP82 agonist, LP82 antagonist, LP82 polynucleotide, LP82 polypeptide, LP82 fragment, LP82 variant, and/or LP82 antibody is also suitably administered by sustained-release systems. Suitable examples of sustained-release compositions include semi-permeable polymer matrices in the form of shaped articles, e.g., films, or microcapsules. Sustained-release matrices include polylactides (U.S. Pat. No. 3,773,919, EP 58,481), copolymers of L-glutamic acid and gamma-ethyl-L-glutamate (Sidman, U. et al., Biopolymers 22:547-556 (1983)), poly (2-hydroxyethyl methacrylate) (R. Langer et al., J. Biomed. Mater. Res. 15:167-277 (1981), and R. Langer, Chem. Tech. 12:98-105 (1982)), ethylene vinyl acetate (R. Langer et al., Id.) or poly-D-(−)-3-hydroxybutyric acid (EP 133,988). Other sustained-release compositions also include liposomally entrapped modified LP82 polypeptides and/or fragments thereof and/or variants thereof. Such liposomes are prepared by methods known per se: DE 3,218,121; Epstein et al., Proc. Natl. Acad. Sci. (USA) 82:3688-3692 (1985); Hwang et al., Proc. Natl. Acad. Sci. (USA) 77:4030-4034 (1980); EP 52,322; EP 36,676; EP 88,046; EDP 143,949; EP 142,641; Japanese Pat. Appl. 83-118008; U.S. Pat. Nos. 4,485,045 and 4,544,545; and EP 102,324. Ordinarily, the liposomes are of the small (about 200-800 Angstroms) unilamellar type in which the lipid content is greater than about 30 mol percent cholesterol, the selected proportion being adjusted for the optimal therapy.

For parenteral administration, in one embodiment, the LP82 agonist, LP82 antagonist, LP82 polynucleotide, LP82 polypeptide, LP82 fragment, LP82 variant, and/or LP82 antibody is formulated generally by mixing it at the desired degree of purity, in a unit dosage injectable form (solution, suspension, or emulsion), with a pharmaceutically acceptable carrier, i.e., one that is non-toxic to recipients' at the dosages and concentrations employed and is compatible with other ingredients of the formulation. For example, the formulation preferably does not include oxidizing agents and other compounds that are known to be deleterious to polypeptides.

Generally, the formulations are prepared by contacting LP82 agonist, LP82 antagonist, LP82 polynucleotide, LP82 polypeptide, LP82 fragment, LP82 variant, and/or LP82 antibody uniformly and intimately with liquid carriers or finely divided solid carriers or both. Then, if necessary, the product is shaped into the desired formulation. Preferably the carrier is a parenteral carrier, more preferably a solution that is isotonic with the blood of the recipient. Examples of such carrier vehicles include water, saline, Ringer's solution, and dextrose solution. Non-aqueous vehicles such as fixed oils and ethyl oleate are also useful herein, as well as liposomes.

The carrier suitably contains minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) polypeptides, e.g., polyarginine or tripeptides; proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, manose, or dextrins; chelating agents such as EDTA; sugar alcohols such as mannitol or sorbitol; counterions such as sodium; and/or nonionic surfactants such as polysorbates, poloxamers, or PEG.

A LP82 agonist, LP82 antagonist, LP82 polynucleotide, LP82 polypeptide, LP82 fragment, LP82 variant, and/or LP82 antibody is typically formulated in such vehicles at a concentration of about 0.1 mg/ml to 100 mg/ml, preferably 1-10 mg/ml, at a pH of about 3 to 8. It will be understood that the use of certain of the foregoing excipients, carriers, or stabilizers will result in the formation of salts of the particular active ingredient(s).

Compositions to be used for therapeutic administration must be sterile. Sterility is readily accomplished by filtration through sterile filtration membranes (e.g., 0.2 micron membranes). Therapeutic compositions generally are placed into a container having a sterile access port, for example, an intravenous solution bag or vial having a stopper piercable by a hypodermic injection needle.

Compositions comprising a LP82 agonist, LP82 antagonist, LP82 polynucleotide, LP82 polypeptide, LP82 fragment, LP82 variant, and/or LP82 antibody ordinarily will be stored in unit or multi-dose containers, for example, sealed ampoules or vials, as an aqueous solution or as a lyophilized formulation for reconstitution. As an example of a lyophilized formulation, 10-ml vials are filled with 5 ml of sterile-filtered 1% (w/v) aqueous solution of one of the LP82 polypeptides and/or fragments and/or LP82 variants of the present invention, and the resulting mixture is lyophilized. The infusion solution is prepared by reconstituting the lyophilized polypeptide using bacteriostatic Water-for-Injection.

The invention also provides a pharmaceutical pack or kit comprising one or more containers filled with one or more of the ingredients of the pharmaceutical compositions of the invention. Associated with such container(s) can be a notice in the form prescribed by a governmental agency regulating the manufacture, use or sale of pharmaceuticals or biological products, which notice reflects approval by the agency of manufacture, use or sale for human administration. In addition, a LP82 agonist, LP82 antagonist, LP82 polynucleotide, LP82 polypeptide, LP82 fragment, LP82 variant, and/or LP82 antibody may be administered in the methods of the present invention in combination with other therapeutic compounds. Administration in combination with one or more further therapeutic agents includes simultaneous (concurrent) and consecutive administration in any order.

The following examples more fully describe the present invention. Those skilled in the art will recognize that the particular reagents, equipment, and procedures described are merely illustrative and are not intended to limit the present invention in any manner.

### EXAMPLES

### Example 1

### LP82 Transgenic Rodent Development

A. Transgene Construction.

Polymerase chain reaction (PCR) primers were synthesized and used to amplify the LP82 coding region from a plasmid containing the full length coding region plus surrounding sequences:

| | |
|---|---|
| 5′-TTGGCGCGCCATCCACCATGAAAGCCTCTAGTC | (SEQ ID NO: 4) |
| TTGCC-3′ | |
| 5′-TAGCGGCCGCTACTTGTCGTCGTCATCCTTGTA | (SEQ ID NO: 5) |
| GTCTTCTGTCTCCTCCATCCATTGCAG-3′ | |

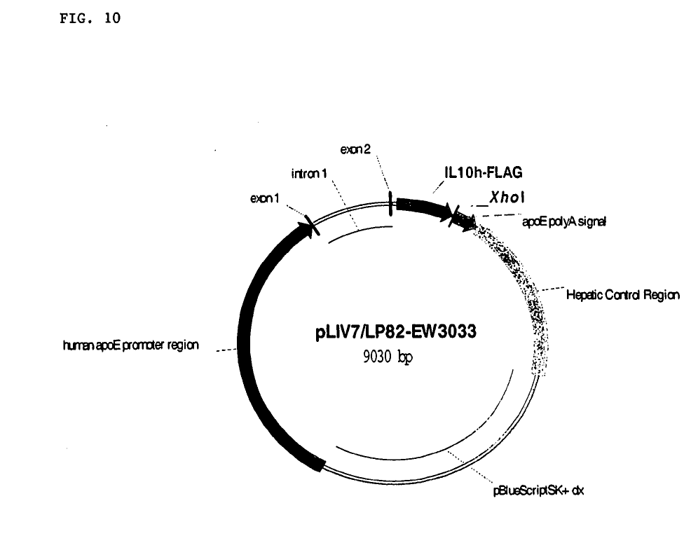

The 5′ oligonucleotide incorporated an Asc I restriction enzyme site and Kozak sequence while the 3′ primer incorporated a Not I restriction enzyme site to facilitate cloning. The amplified ˜0.7 kb fragment was ligated into the multiple cloning site (Asc 1-Not I) of plasmid pK409, a derivative of the pFastBac expression vector (Gibco BRL). The constructed vector with LP82Flag [pEW1943] was subsequently digested with Asc 1-Xho I and cloned into pLIV7 (provided by John Taylor, J. David Gladstone Institutes) at the Mlu I and Xho I sites generating plasmid pLIV7/LP82Flag-pEW3033 (see FIG. 10).

B. Transgenic Animal Development.

Transgenic mice were generated using established techniques (see e.g., Hogan, B. et al. (1986) Manipulating the Mouse Embryo: A Laboratory Manual. Cold Spring Harbor Laboratory, NY] as modified by Fox and Solter (Mol. Cell. Biol. 8:5470, 1988). Briefly, a 6.4 kb DNA fragment encompassing the human apolipoprotein E (hApoE) gene promoter-5′ hApoE untranslated region-LP82/FLAG-hepatic control region (HCR) fusion gene was excised from plasmid pLIV7-LP82 by digestion with Sal I and Spe I and purified by gel electrophoresis and glass bead extraction. The purified DNA fragment encompassing the hApoE gene promoter-5′ hApoE untranslated region-LP82-HCR fusion gene was microinjected into the male pronuclei of newly fertilized one-cell-stage embryos (zygotes) of the FVB/N strain. The embryos were cultured in vitro overnight to allow development to the two-cell-stage. Two-cell embryos were then transplanted into the oviducts of pseudopregnant ICR strain mice to allow development to term. To test for the presence of the transgene in the newborn mice, a small piece of toe was removed from each animal and digested with proteinase K to release the nucleic acids. A sample of the toe extract was subsequently subjected to PCR analysis using primers specific for the hApoE untranslated region to identify transgene containing mice. Five founder transgenic mice were identified designated 14074, 14076, 14282, 14283 and 14379. Each of these founders was bred to produce F1 and F2 progeny.

The runts of the litters were LP82 transgenic mice. The normal sized pups were removed from the litter to allow for better survival of the LP82 transgenic mice. The LP82 transgenic mice fed well and caught up to wild type in size about four weeks after birth.

### Example 2

### Biological Testing for Anti-Obesity Molecules

Various animal models of obesity and diabetes are known in the art and generally accepted as being indicative of the obese or diabetic condition (for example see U.S. Pat. No. 5,597,797).

One biological test model is to inject a putative LP82 agonist or LP82 antagonist or a test LP82 polynucleotide, LP82 polypeptide, LP82 fragment, LP82 variant, and/or LP82 antibody by any of several routes of administration (e.g. i.v., s.c., i.p., or by minipump or cannula) and then to monitor food and water consumption, body weight gain, plasma chemistry or hormones (glucose, insulin, ACTH, corticosterone, GH, T4) over various time periods. Suitable test animals include normal mice (ICR, etc.) and obese mice (ob/ob, Avy/a, KK-Ay, tubby, fat) as well as LP82 transgenic mice similar to those described in Example 1. Controls for non-specific effects for these injections are done using vehicle with or without the active agent of similar composition in the same animal monitoring the same parameters. Proteins demonstrating activity in these models will demonstrate similar activity in other mammals, particularly humans.

A similar model is to inject the test molecule directly into the brain (e.g. i.c.v injection via lateral or third ventricles, or directly into specific hypothalamic nuclei (e.g. arcuate, paraventricular, periformical nuclei). The same parameters as above could be measured, or the release of neurotransmitters that are known to regulate feeding or metabolism could be monitored (e.g. NPY, galanin, norepinephrine, dopamine, b-endorphin release).

If the compounds are active in at least one of the above biological tests they can be considered as anti-obesity agents useful in the methods of the present invention. As such, they are useful in treating or preventing obesity and those disorders implicated by obesity. Furthermore, the compounds are useful for controlling weight for cosmetic purposes in mammals. A cosmetic purpose seeks to control the weight of a mammal to improve bodily appearance. The mammal is not necessarily obese. Such cosmetic use forms part of the present invention.

### Example 3

### ; Dietary Experiments with LP82 Transgenic Mice

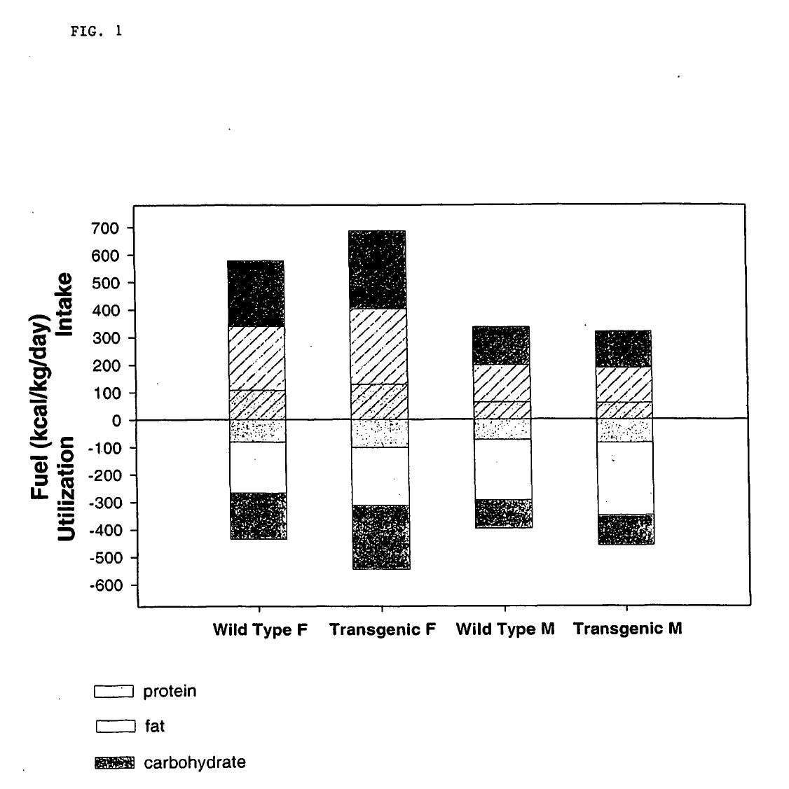

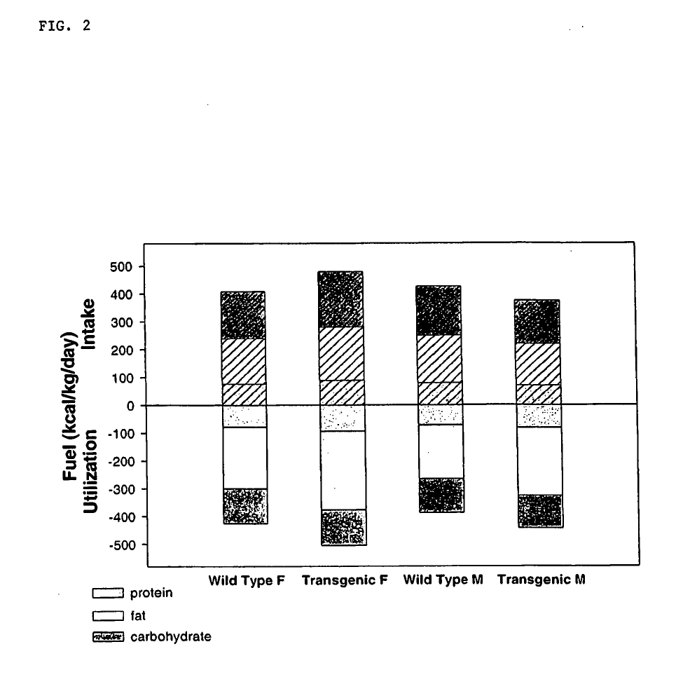

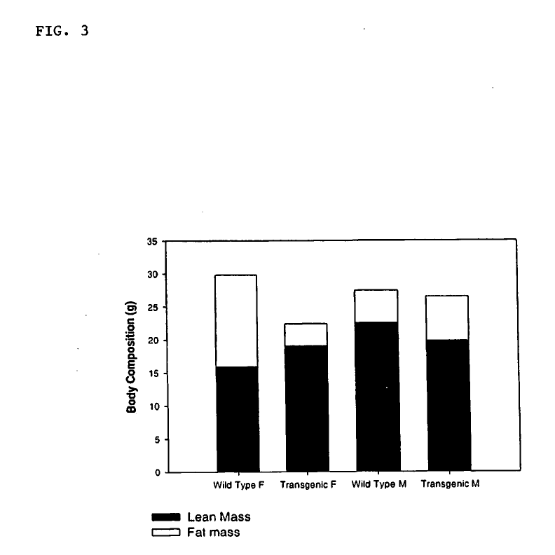

Wild type (4 animals per group) and transgenic mice (3 animals per group) of each gender were weaned onto a diet comprised of 18% protein, 26% fat, and 56% carbohydrate. The mice were switched to a diet comprised of 19% protein, 40% fat, and 41% carbohydrate at about 8-weeks of age and the mice were transferred to a metabolic physiology lab. After 27 and 43 days on this diet, the mice were placed in an indirect calorimeter (Oxymax, Columbus Instruments; Columbus, Ohio) for 24 hours. Wild-type females and both male genotypes gained body weight during this period while transgenic female mice appeared to resist such weight gain. Although the transgenic females appeared to consume more food than did their wild-type counterparts (average values per group shown in FIG. 1), this was coincident with enhanced caloric expenditure, especially that of carbohydrate. The transgenic males tended to utilize more carbohydrate than the male wild-type controls but intake was comparable with both sets of animals. Similar data were obtained after consumption of this diet for another 16 days (FIG. 2). Because the transgenic females utilized more fat for fuel when switched to a diet that was rich in fat and carbohydrate, those mice gained less fat mass during this dietary challenge (FIG. 3). Male transgenic mice appeared to gain more fat mass than the male control mice despite that they appeared to utilize fat fuel to a greater extent.

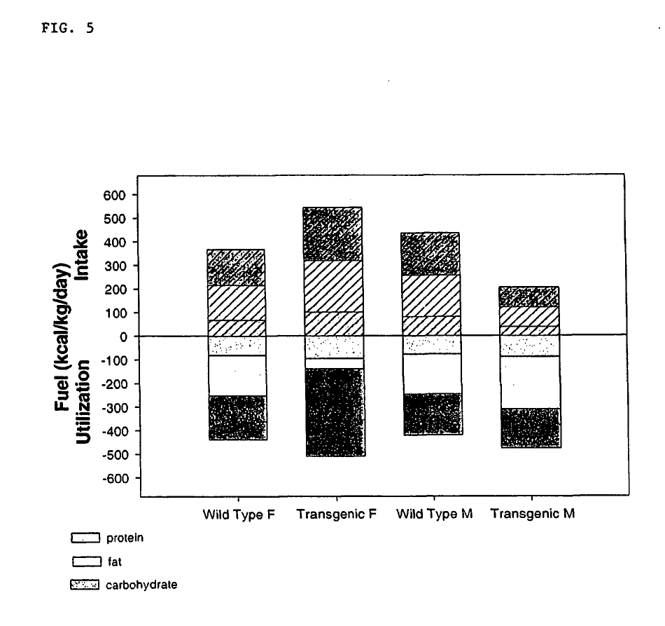

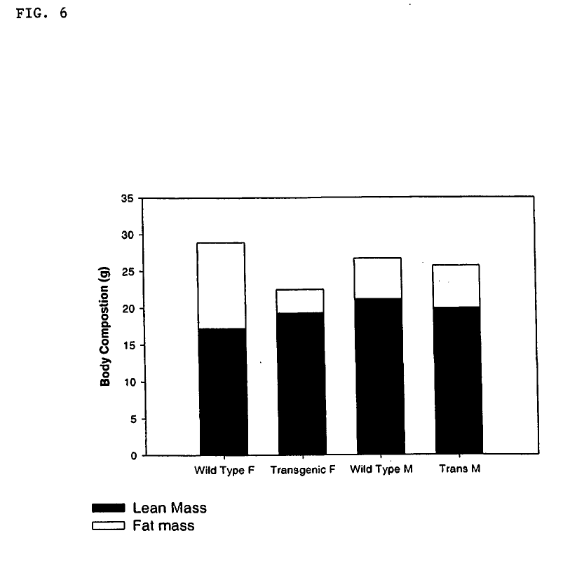

All mice were then offered a high carbohydrate diet comprised of 22% protein, 5% fat, and 73% carbohydrate. Male transgenic mice lost weight when placed on this diet but no changes in body weight were observed for either female genotype. Transgenic males over-expressing LP82 consumed less food as measured after one week on this diet (FIG. 4) or after two weeks (FIG. 5) then did the control mice. No compensatory decrease in energy expenditure was observed despite that caloric intake was decreased. Such a negative energy balance was associated with increases in lipid utilization. In contrast, female transgenics appeared to remain in energy balance by matching an enhanced caloric intake with increased energy expenditure. The latter was increased at the expense of carbohydrate utilization. Thus, there was a trend for only the male transgenic mice to lose fat mass when placed on this high carbohydrate diet (FIG. 6). Females and male wild-type mice maintained their body composition throughout this dietary challenge.

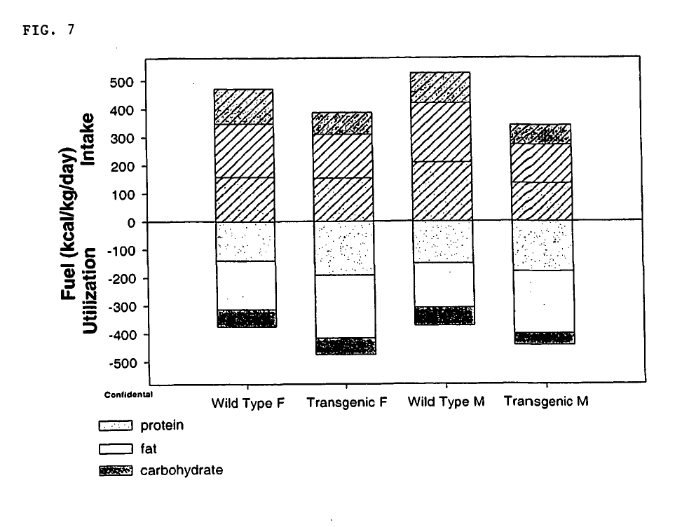

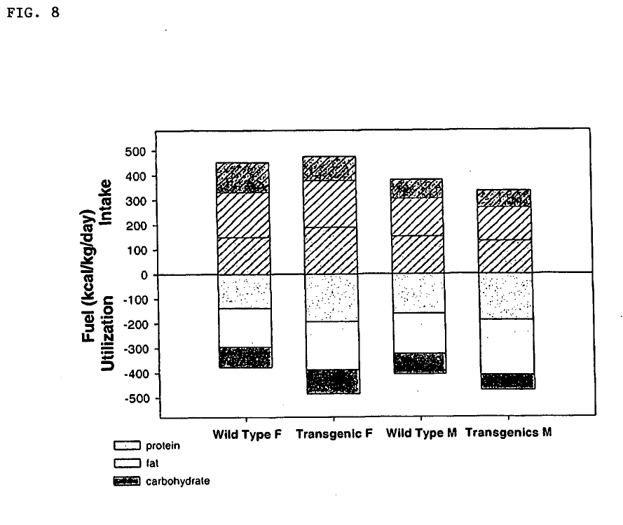

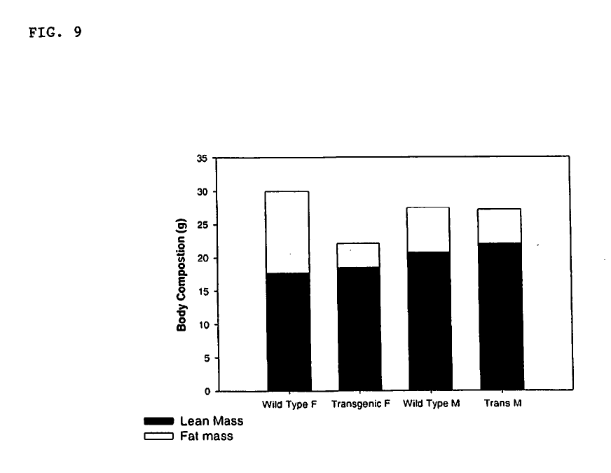

The mice were then offered a diet rich in protein (40%) and fat (40%) with 20% carbohydrate for 20 days. Transgenic males and females entered a negative energy balance after 1 week on this diet. This was a consequence of decreased feeding without a compensatory decrease in caloric expenditure (FIG. 7). In fact, the latter tended to be increased when compared to that of the respective wild-type mice. A greater caloric expenditure than caloric intake was maintained for an additional 15 days in the transgenic mice (FIG. 8). This negative energy balance was greater for males than females and thus a decrease in fat mass was observed in the transgenic males (FIG. 9).

We claim:

1. A method of inhibiting fat accretion in a mammal by administration of a therapeutically effective amount of an LP82 polynucleotide, LP82 polypeptide, LP82 variant, and/or LP82 antibody.

2. A method of treating or preventing body weight disorders including, but not limited to, obesity, cachexia, bulimia, anorexia, and/or disorders commonly associated with these conditions comprising the administration to a mammal in need of such treatment a therapeutically effective amount of a pharmaceutical composition comprising a LP82 agonist, LP82 antagonist, LP82 polynucleotide, LP82 polypeptide, LP82 variant, and/or LP82 antibody.

3. A method of prophylactically preventing fat accretion or maintaining a desired body mass composition in a subject having a substantially normal body mass composition comprising the step of administering pharmaceutical composition comprising a LP82 agonist, LP82 antagonist, LP82 polynucleotide, LP82 polypeptide, LP82 variant, and/or LP82 antibody.

4. The method of claim 3 wherein said LP82 polypeptide is selected from the group consisting of:
a) a polypeptide comprising residues from about 1 through about 176 of SEQ ID NO:2.
b) a polypeptide comprising residues from about 15 through about 176 of SEQ ID NO:2;
c) a polypeptide comprising residues from about 25 through about 176 of SEQ ID NO: 2;
d) a polypeptide comprising residues from about 50 through about 176 of SEQ ID NO:2.
e) a polypeptide comprising residues from about 70 through about 176 of SEQ ID NO:2;
f) a polypeptide comprising residues from about 90 through about 176 of SEQ ID NO: 2.
g) a polypeptide comprising residues from about 25 through about 160 of SEQ ID NO:2;
h) a polypeptide comprising residues from about 25 through about 140 of SEQ ID NO:2;
i) a polypeptide comprising residues from about 25 through about 120 of SEQ ID NO:2; and
j) a polypeptide comprising residues from about 25 through about 100 of SEQ ID NO:2.

5. A pharmaceutical compound comprising one or more pharmaceutically acceptable diluents, carriers, or excipients and at least one LP82 polypeptide according to claim 4.

6. A composition comprising a LP82 agonist, LP82 antagonist, LP82 polynucleotide, LP82 polypeptide, LP82 variant, and/or LP82 antibody for use as a medicament to prevent or treat body weight disorders including, but not limited to, obesity, cachexia, bulimia, anorexia, and/or disorders commonly associated with such conditions including, but not limited to, elevated serum glucose level, an elevated serum lipid level, or an elevated serum free fatty acid level.

7. The use of a LP82 agonist, LP82 antagonist, LP82 polynucleotide, LP82 polypeptide, LP82 variant, and/or LP82 antibody for the manufacture of a medicament for preventing or treating body weight disorders including, but not limited to, obesity, cachexia, bulimia, anorexia, and/or disorders commonly associated with such conditions including, but not limited to, elevated serum glucose level, an elevated serum lipid level, or an elevated serum free fatty acid level.

8. A method of increasing thermogenesis in mammal, comprising administering to said mammal a nucleic acid molecule which encodes LP82, wherein said administering of said nucleic acid molecule increases the level of LP82 expression in one or more tissues of said subject.

9. A method of treating obesity in a mammal, comprising administering to said mammal a nucleic acid molecule which encodes LP82, wherein said administering of said nucleic acid molecule increases the level of LP82 expression in one or more tissues of said subject.

10. A method of decreasing the amount of fat in a mammal comprising administering to said mammal a nucleic acid molecule which encodes LP82, wherein said administering of said nucleic acid molecule increases the level of LP82 expression in one or more tissues of said subject.

11. A method of treating diabetes in a mammal, comprising administering to said mammal a nucleic acid molecule which encodes LP82, wherein said administering of said nucleic acid molecule increases the level of LP82 expression in one or more tissues of said subject.

12. A method of decreasing thermogenesis in a mammal comprising administering to said mammal an LP82 polynucleotide wherein said administration of said polynucleotide decreases the level of LP82 polypeptide expression in one or more tissues of said mammal.

13. The method of claim 12, wherein said subject has a susceptibility to obesity.

14. A transgenic mouse all of whose germ cells and somatic cells contain one or more recombinant constructs of an LP82 polynucleotide operably linked to a promoter and said one or more recombinant constructs having been introduced into said mouse, or an ancestor of said mouse, at an embryonic stage.
